(19) United States
(12) Patent Application Publication
(10) Pub. No.: US 2024/0327791 A1
(43) Pub. Date: Oct. 3, 2024

(54) METHODS OF T CELL PRODUCTION

(71) Applicant: Adaptimmune Limited, Abingdon, Oxfordshire (GB)

(72) Inventor: Cheng-Tao YANG, Abingdon, Oxfordshire (GB)

(21) Appl. No.: 18/291,856

(22) PCT Filed: Aug. 3, 2022
(86) PCT No.: PCT/EP2022/071867

§ 371 Date: Jan. 24, 2024

(30) Foreign Application Priority Data
Aug. 3, 2021 (GB) 2111169.5

(51) Int. Cl. C12N 5/0783; A61K 35/17; A61K 39/00; C07K 14/725; C12N 15/86

(52) U.S. Cl. CPC C12N 5/0636; A61K 35/17; A61K 39/4611; A61K 39/4632; A61K 39/464499; C07K 14/7051; C12N 15/86; C12N 2501/125; C12N 2501/145; C12N 2501/2307; C12N 2501/26; C12N 2501/999; C12N 2510/00; C12N 2740/15043

(57) ABSTRACT

This invention relates to methods of producing a population of progenitor T cells comprising differentiating haematopoietic progenitor cells (HPCs) into progenitor T cells in the presence of a pyrimidoindole compound, such as methyl 4-(3-piperidin-1-ylpropylamino)-9H-pyrimido[4,5-b] indole-7-carboxylate (UM729) or (1R,4R)-N1-(2-benzyl-7-(2-methyl-2H-tetrazol-5-yl)-9H-pyrimido[4,5-b]indol-4-yl)cyclohexane-1,4-diamine (UM171). The progenitor T cells may be matured, activated and expanded, for example for use in immunotherapy.

### FIELD

This invention relates to the production of T cells, for example for use in immunotherapy.

### BACKGROUND

Immunotherapeutics are poised to transform the cancer treatment landscape with the promise of long-term survival (McDermott et al., Cancer Treat Rev. 2014 October; 40(9): 1056-64). There is a clear unmet medical need for new immunomodulatory drugs to expand patient population and range of tumor types. In addition, new agents are needed to enhance the magnitude and duration of anti-tumor responses. The development of these agents has been possible because of the in-depth understanding of the basic principles controlling T-cell immunity over the last two decades (Sharma and Allison, Cell. 2015 Apr. 9; 161(2): 205-14). This typically requires tumor specific CD4+ and CD8+ T-cells recognising tumor-associated peptide antigens presented by MHC molecules. Different vaccination strategies and adoptive transfer of ex vivo expanded tumor infiltrated lymphocytes have in some cases demonstrated the ability of tumor specific T-cells to treat late-stage cancer (Rosenberg et al., Nat Med. 2004 September; 10(9): 909-15).

However, current adoptive T cell therapies are limited by a lack of suitable patient and tumor-specific T cells and there is a need for therapeutically sufficient and functional antigen-specific T cells for effective use in immunotherapy.

Pyrimido[4,5-b] indole derivatives have been reported to enhance the expansion of haematopoietic stem cells (HSCs) but to inhibit their differentiation (WO2013110198).

### SUMMARY

The present inventors have recognised that certain pyrimidoindole compounds, such as UM729 and UM171, may be used to increase the differentiation of haematopoietic progenitor cells into progenitor T cells and/or the expansion of populations of progenitor T cells. This may be useful for example, in the production of T cells for immunotherapy.

A first aspect of the invention provides a method of producing a population of progenitor T cells comprising;
differentiating a population of haematopoietic progenitor cells (HPCs) into progenitor T cells in the presence of a pyrimidoindole compound.

The presence of the pyrimidoindole compound may increase the proportion of HPCs that differentiate into progenitor T cells.

A method of the first aspect may further comprise expanding the population of progenitor T cells. Preferably, the population of progenitor T cells is expanded by culturing in the presence of a pyrimidoindole compound.

A second aspect of the invention provides a method of expanding a population of progenitor T cells comprising culturing a population of progenitor T cells in the presence of a pyrimidoindole compound.

The presence of the pyrimidoindole compound may increase the number of progenitor T cells in the population following said culturing.

A third aspect of the invention provides a method of producing a population of T cells comprising;
producing a population of progenitor T cells by a method of the first or second aspect,
maturing the progenitor T cells to produce a population of TCR αβ+ T cells and;
optionally activating and expanding the TCR αβ+ T cells to produce a population of TCR αβ+CD8+ T cells or a population of TCR αβ+CD4+ T cells.

A fourth aspect of the invention provides a population of progenitor T cells produced by a method of the first or second aspect or a population of TCR αβ+ T cells produced by a method of the third aspect.

A fifth aspect of the invention provides a pharmaceutical composition comprising a population of TCR αβ+ T cells of the third aspect and a pharmaceutically acceptable excipient.

A sixth aspect of the invention provides a method of treatment comprising administering a therapeutically effect amount of a population of TCR αβ+ T cells of the third aspect to an individual in need thereof.

A seventh aspect of the invention provides the use of a pyrimidoindole compound in the differentiation of HPCs into progenitor T cells.

An eighth aspect of the invention provides the use of a pyrimidoindole compound to expand a population of progenitor T cells.

Other aspects and embodiments of the invention are described in more detail below.

### BRIEF DESCRIPTION OF THE FIGURES

FIG. 1 shows a schematic view of an example of a six-stage method for generating T cells from iPSCs.

FIG. 2A shows that haematopoietic progenitor cells maintain viability when treated over 21 days with 0.25 μM to 1.0 μM UM729. FIG. 2B shows that the viability and proliferation of haematopoietic progenitor cells is reduced or ceases when treated over 21 days with 5.0 μM UM729 or higher.

FIG. 3 shows that treatment of haematopoietic progenitor cells treated over 21 days with 0.25 μM to 1.0 μM UM729 increases the lymphoid fraction and reduces the myeloid fraction of the cells.

FIG. 4A shows that treatment of haematopoietic progenitor cells over 21 days with 0.25 μM to 1.0 μM UM729 promotes differentiation into iProT cells (which are CD7+, CD5+ and CD34−). FIG. 4B shows that treatment of haematopoietic progenitor cells over 21 days with 0.25 μM to 1.0 μM UM729 enriches the percentage of iProT cells (which are CD7+, CD5+ and CD34−).

FIGS. 5A and 5B show that treatment of haematopoietic progenitor cells over 21 days in stage 4 medium with 0.25 μM to 1.0 μM UM729 decreases the fraction of CD8+CD4+ double positive cells.

FIG. 6 shows that haematopoietic progenitor cells maintain viability when treated over 35 days in stage 4 medium with 0.25 μM to 1.0 μM UM729 FIG. 7 shows that treatment of haematopoietic progenitor cells over 35 days in stage 4 medium with 0.25 μM to 1.0 μM UM729 promotes differentiation into iProT cells (which are CD7+, CD5+ and CD34−).

FIG. 8 shows that treatment of haematopoietic progenitor cells over 35 days in stage 4 medium with 0.25 μM to 1.0 μM UM729 decreases the fraction of CD8+CD4+ double positive cells.

FIG. 9 shows that treatment in stage 4 medium with 0.25 μM to 1.0 μM UM729 greatly enhances the expansion of iProT cells

FIG. 10 shows that cells pre-treated with UM729 for 21 days in stage 4 medium will become CD4+CD8+ double positive if grown for a further 14 days in stage 5 medium in the absence of UM729.

FIG. 11 shows that the addition of UM729 to stage 4 media improved the recovery of iPro T cells from freeze/thaw, Cells were stained with solution 6 (PI staining nonviable cells only and VB-48™ which stains cells in an intensity-dependent manner reflecting their redox status).

FIG. 12 shows that cells pre-treated with UM729 at 1 μM in the stage 4 medium presented a higher viability (12A) and an increased cell proliferation (12B) after culture in stage 5 medium for 7 days.

FIG. 13 shows the viability (13A) and proliferation (13B) of iPSC derived CD34+ cells (HPCs) cultured in stage 4 medium supplemented with different concentrations of UM171 for 21 days FIG. 14 shows the proportion of CD45+CD34+CD7+(common lymphoid progenitors) following culture of iPSC derived CD34+ cells (HPCs) in stage 4 medium supplemented with different concentrations of UM171 for 21 days.

FIG. 15 shows the proportion of CD45+CD5+CD7+ progenitor T cells (15A), CD4+CD8+DP T cells and CD4-CD8+SP T cells (15B) after 14 days culture in stage 5 T cell maturation medium. following culture of iPSC derived CD34+ cells (HPCs) in stage 4 medium supplemented with different concentrations of UM171 for 21 days in stage 4.

### DETAILED DESCRIPTION

This invention relates to methods of producing a population of progenitor T cells that comprise differentiating haematopoietic progenitor cells (HPCs) into progenitor T cells in the presence of a pyrimidoindole compound. This invention also relates to methods of expanding populations of progenitor T cells by culturing them in the presence of a pyrimidoindole compound. These methods may be useful in the generation of immune cells, for example T cells, including allogeneic T cells, for use in immunotherapy and other applications.

The presence of pyrimidoindole compound during the differentiation of haematopoietic progenitor cells (HPCs) into T cell progenitors may increase the amount or proportion of progenitor T cells in the population of differentiated cells relative to the absence of the pyrimidoindole compound during the differentiation. For example, the amount or proportion of CD34-CD5+CD7+ progenitor T cells in the population following differentiation may be increased in the presence of pyrimidoindole compound.

The presence of pyrimidoindole compound during the culturing of progenitor T cells may increase the proliferation of the progenitor T cells while inhibiting further differentiation of the progenitor T cells, leading to increased proportion of such T cells in the population. For example, the amount or number, as well as percentage, of CD5+CD7+ progenitor T cells following culture in the presence of pyrimidoindole compound may be increased. In addition, the presence of a pyrimidoindole compound during culturing of the progenitor T cells can improve the recovery from freeze/thaw of T cells in subsequent differentiation steps.

T cells (also called T lymphocytes) are white blood cells that play a central role in cell-mediated immunity. T cells can be distinguished from other lymphocytes by the presence of a T cell receptor (TCR) on the cell surface. There are several types of T cells, each type having a distinct function. T helper cells (T_{H} cells) are known as CD4⁺ T cells because they express the CD4 surface glycoprotein. CD4⁺ T cells play an important role in the adaptive immune system and help the activity of other immune cells by releasing T cell cytokines and helping to suppress or regulate immune responses. They are essential for the activation and growth of CD4+CD8+ T cells. CD4+CD8+ T cells (Tc cells, CTLs, killer T cells, CD4+CD8+ T cells) are known as CD8⁺ T cells because they express the CD8 surface glycoprotein. CD8⁺ T cells act to destroy virus-infected cells and tumour cells. Most CD8⁺ T cells express TCRs that can recognise a specific antigen displayed on the surface of infected or damaged cells by a class I MHC molecule. Specific binding of the TCR and CD8 glycoprotein to the antigen and MHC molecule leads to T cell-mediated destruction of the infected or damaged cells.

T cell receptors (TCRs) may bind specifically to major histocompatibility complexes (MHC) on the surface of cells that display a peptide fragment of a target antigen. For example, TCRs may bind specifically to a major histocompatibility complex (MHC) on the surface of cancer cells that displays a peptide fragment of a tumour antigen. Alternatively, TCRs may recognise specific antigen or peptide thereof independent of presentation by MHC. T cells comprising such TCRs may be produced according to the methods of the present invention. An MHC is a set of cell-surface proteins which allow the acquired immune system to recognise ‘foreign’ molecules. Proteins are intracellularly degraded and presented on the surface of cells by the MHC. MHCs displaying ‘foreign’ peptides, such a viral or cancer associated peptides, are recognised by T cells with the appropriate TCRs, prompting cell destruction pathways. MHCs on the surface of cancer cells may display peptide fragments of tumour antigen i.e. an antigen which is present on a cancer cell but not the corresponding non-cancerous cell. T cells which recognise these peptide fragments may exert a CD4+CD8+ effect on the cancer cell.

Progenitor T cells are multi-potent lymphopoietic progenitor cells that are capable of giving rise to αβ+ T cells, γδ+ T cells, tissue resident T cells and NKT cells. Progenitor T cells may commit to the as T cell lineage after pre-TCR selection in the thymus (Trotman-Grant et al., Nat. Commun. 2021 12 (1) 5023; Kennedy et al., Cell Rep 2012 2(6) 1722-1735). Progenitor T cells may be capable of in vivo thymus colonization and may be capable of committing to the αβ T cell lineage after pre-TCR selection in the thymus. Progenitor T cells may also be capable of maturation into cytokine-producing CD3⁺ T-cells.

Progenitor T cells may express CD5 and CD7 i.e. the progenitor T cells may have a CD5+CD7+ phenotype. Progenitor T cells may also co-express CD44, CD25 and CD2. For example, progenitor T cells may have a CD5+, CD7+CD44+, CD25+CD2+ phenotype. Progenitor T cells may also co-express CD45. Progenitor T cells may lack expression, for example cell surface expression, of CD3, CD4 and CD8. Progenitor T cells may lack expression, for example cell surface expression, of CD34 (i.e. progenitor T cells may have a CD34-phenotype).

Populations of progenitor T cells (also called iPro T cells herein) are produced as described herein by the directed differentiation of haematopoietic progenitor cells (HPCs). HPCs (also called hematopoietic stem and progenitor cells or HSPCs) are multipotent stem cells that are committed to a hematopoietic lineage and are capable of further hematopoietic differentiation into all blood cell types including myeloid and lymphoid lineages, including monocytes, B cells, NK cells, NKT cells, TILs and T cells. HPCs may express CD34. HPCs may co-express CD45. HPCs may also co-express CD117, CD133, CD45 and FLK1 (also known as KDR or VEGFR2). HPCs may be negative for expression of CD38 and other lineage specific markers. For example, HPCs may display one or more, preferably all of CD34+CD133+CD45+ FLK1+CD38−.

In some embodiments, HPCs may be differentiated into progenitor T cells through a common lymphoid progenitor intermediate. For example, HPCs may be differentiated into common lymphoid progenitors (CLPs) that subsequently differentiate into progenitor T cells. CLPs are multipotent stem cells that are committed to a lymphoid lineage (Hoebeke et al., Leukemia 2007 21(2) 311-319). CLPs may express CD34. CLPs may co-express CD45. CLPs may also co-express CD7. CLPs may also co-express CD45RA and HLA-DR. CLPs may be negative for expression of CD38, CD5 and other markers, such as c-kit and Thy-1 For example, CLPs may display one or more, preferably all of CD34+CD45+CD7+, CD38− and CD5−.

Haematopoietic progenitor cells (HPCs) may be differentiated into progenitor T cells by culturing the population of HPCs in the presence of a pyrimidoindole compound under suitable conditions to promote lymphoid differentiation. For example, the haematopoietic progenitor cells may be cultured in a lymphoid expansion medium supplemented with the pyrimidoindole compound.

A pyrimidoindole compound for use in the present invention may have a pyrimidoindole core which is typically substituted on the pyrimidine and the benzene rings of the core.

Preferably, the pyrimidoindole compound is a pyrimido[4,5-b]indole, which is optionally substituted, and is preferably substituted. For example, the pyrimidoindole compound may be a pyrimido[4,5-b]indole compound of formula (I) to (VI) as described in U.S. Ser. No. 10/336,747B2 and WO2013110198.

Following standard naming conventions, the nitrogen ring atoms of the pyrimidine are located at the 1- and 3-positions of the pyrimido[4,5-b]indole core, and the nitrogen ring atom of the indole is located at the 9-position.

The pyrimido[4,5-b]indole may be substituted, and is preferably substituted with two or three substituents. These substituents are typically provided to the pyrimidine and the benzene rings of the pyrimido[4,5-b]indole, as described below. The pyrimidine ring of the pyrimido[4,5-b]indole compound may be monosubstituted or disubstituted. The pyrimidine ring may be substituted at one or both of the 2- and the 4-positions. Preferably, the pyrimidine is substituted at least at the 4-position, and optionally at the 2-position. Preferably, the 2-position is unsubstituted.

The pyrimido[4,5-b]indole compound may have the general Formula I, II, III, or IV;

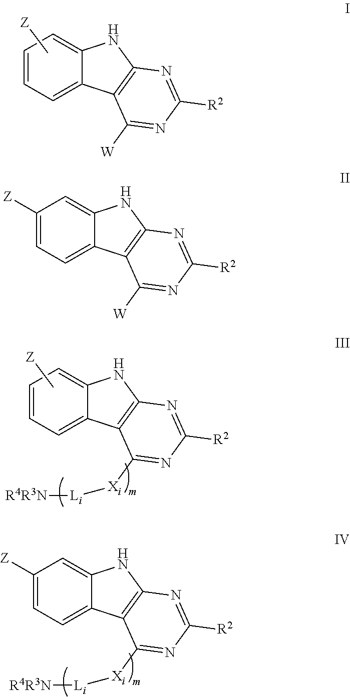

The substituents in Formulae I, II, III and IV are defined below.

Z is: 1) —P(O) (OR¹) (OR¹), 2) —C(O)OR¹, 3) —C(O)NHR¹, 4) —C(O)N(R¹)R¹, 5) —C(O)R¹, 6) —CN, 7) —SR¹, 8) —S(O)₂NH₂, 9) —S(O)₂NHR¹, 10) —S(O)₂N(R¹)R¹, 11) —S(O)R¹, 12) —S(O)₂R¹, 13) -L, 14) -benzyl optionally substituted with 1, 2 or 3 R^{A} or R¹ substituents, 15) -L-heteroaryl optionally substituted with one or more R^{A} or R¹ substituents attached on either or both the L and the heteroaryl groups, 16) -L-heterocyclyl optionally substituted with one or more RA or R¹ substituents attached on either one or both the L and the heterocyclyl groups, 17) -L-aryl optionally substituted with one or more R^{A} or R¹ substituents attached on either or both the L and the aryl groups, 18) -heteroaryl optionally substituted with one or more R^{A} or R¹ substituents, or 19) -aryl optionally substituted with one or more R^{A} or R¹ substituents. In this list, each substituent is optionally attached to the L group if it is not already present; and, when (R¹) and R¹ are attached to a nitrogen atom, optionally they join together with the nitrogen atom to form a 3 to 7-membered ring which optionally includes one or more other heteroatom selected from N, O and S, optionally the ring is substituted with one or more R¹ or R^{A}.

W is H, a halogen or a group that is attached to the pyrimido indole core of the molecule through an atom which is N, O, S, or C. Optionally, W comprises at least one moiety which is saturated, unsaturated, linear, branched and/or cyclic alkyl and/or heteroalkyl having 1 to 20 carbon atoms. Also, optionally, the moiety comprises at least one other hetero atom which is N, O or S.

More specifically, W is 1) —H, 2) -halogen, 3) —OR¹, 4) -L-OH, 5) -L-OR¹, 6) —SR¹, 7) —CN, 8) P(O)(OR¹)(OR¹), 9) NHR¹, 10) —N(R¹)R¹, 11) -L-NH₂, 12) -L-NHR¹, 13) -L-N(R¹)R¹, 14) -L-SR¹, 15) -L-S(O)R¹, 16) -L-S(O)₂R¹, 17) -L-P(O)(OR¹)(OR¹), 18) —C(O)OR¹, 19) —C(O)NH₂, 20) —C(O)NHR¹, 21) —C(O)N(R¹)R¹, 22) —NHC(O)R¹, 23) —NR¹C(O)R¹, 24) —NHC(O)OR¹, 25) —NR¹C(O)OR¹, 26) —OC(O)NH₂, 27) —OC(O)NHR¹, 28) —OC(O)N(R¹)R¹, 29) —OC(O)R¹, 30) —C(O)R¹, 31) —NHC(O)NH₂, 32) —NHC(O)NHR¹, 33) —NHC(O)N(R¹)R¹, 34) —NR¹C(O)NH₂, 35) —NR¹C(O)NHR¹, 36) —NR¹C(O)N(R¹)R¹, 37) —NHS(O)₂R¹, 38) —NR'S(O)₂R¹, 39) —S(O)₂NH₂, 40) —S(O)₂NHR¹, 41) —S(O)₂N(R¹)R¹, 42) —S(O)R¹, 43) —S(O)₂R¹, 44) —OS(O)₂R¹, 45) —S(O)₂OR¹, 46) -benzyl optionally substituted with 1, 2 or 3 R^{A} or R¹ substituents, 47) -L-heteroaryl optionally substituted with one or more R^{A} or R¹ substituents attached on either or both the L and heteroaryl groups, 48) -L-heterocyclyl optionally substituted with one or more R^{A} or R¹ substituents attached on either one or both the L and heterocyclyl groups, 49) -L-aryl optionally substituted with one or more R^{A} or R¹ substituents attached on either or both the L and the aryl groups, 50) -L-NR¹(R¹), 51) -(L-)₂NR¹, 52) -L-(N(R¹)-L)ₙ-N(R¹)R¹, 53) -L-(N(R¹)-L)ₙ-heteroaryl optionally substituted with one or more R^{A} or R¹ substituents attached on either or both the L and heteroaryl groups, 54) -L-(N(R¹)-L)ₙ-heterocyclyl optionally substituted with one or more R^{A} or R¹ substituents attached on either or both the L and heterocyclyl groups, 55) L-(N(R¹)-L)ₙ-aryl optionally substituted with one or more RA or R¹ substituents attached on either or both the L and aryl groups, 56) —O-L-N(R¹)R¹, 57) —O-L-heteroaryl optionally substituted with one or more R^{A} or R¹ substituents attached on either or both the L and the heteroaryl groups, 58) —O-L-heterocyclyl optionally substituted with one or more R^{A} or R¹ substituents attached on either or both the L and the heterocyclyl groups, 59) —O-L-aryl optionally substituted with one or more RA or R¹ substituents attached on either or both the L and the aryl groups, 60) —(O-L)₂-NR¹, 61) —O-L-(N(R¹)-L)ₙ-N(R¹)R¹, 62) —O-L-(N(R¹)-L)ₙ-heteroaryl optionally substituted with one or more R^{A} or R¹ substituents attached on either or both the L and heteroaryl groups, 63) —O-L-(N(R¹)-L)ₙ-heterocyclyl optionally substituted with one or more R^{A} or R¹ substituents attached on either or both the L and heterocyclyl groups, 64) —O-L-(N(R¹)-L)ₙ-aryl optionally substituted with one or more R^{A} or R¹ substituents attached on either or both the L and aryl groups, 65) —S-L-heteroaryl optionally substituted with one or more RA or R¹ substituents attached on either or both the L and heteroaryl groups, 66) —S-L-heterocyclyl optionally substituted with one or more R^{A} or R¹ substituents attached on either or both the L and heterocyclyl groups, 67) —S-L-aryl optionally substituted with one or more R^{A} or R¹ substituents attached on either or both the L and aryl groups, 68) —(S-L)₂-NR¹, 69) —S-L-(N(R¹)-L)ₙ-N(R¹)R¹, 70) —S-L-(N(R¹)-L)ₙ-heteroaryl optionally substituted with one or more R^{A} substituents, 71) —S-L-(N(R¹)-L)ₙ-heterocyclyl optionally substituted with one or more R^{A} substituents, 72) —S-L-(N(R¹)-L)ₙ-aryl optionally substituted with one or more R^{A} substituents, 73) —N(R¹)R¹, 74) —(N(R¹)-L)ₙ-N(R¹)R¹, 75) —(N(R¹)-L)₂-NR¹, 76) —(N(R¹)-L)ₙ-N(R¹)R^{A}, 77) —(N(R¹)-L)ₙ-heteroaryl optionally substituted with one or more R^{A} or R¹ substituents, 78) —(N(R¹)-L)ₙ-heterocyclyl optionally substituted with one or more R^{A} or R¹ substituents, 79) —(N(R¹)-L)ₙ-aryl optionally substituted with one or more R^{A} or R¹ substituents, 80) -heteroaryl optionally substituted with one or more R^{A} substituents, or 81) -aryl optionally substituted with one or more R^{A} substituents. In this list, each substituent is optionally attached to the L group if it is not already present; and, when two R¹ substituents are present on the same nitrogen atom, then each R¹ substituent is independently selected from the list of R¹ values described herein; and n is an integer equal to either 0, 1, 2, 3, 4 or 5; and when (R¹) and R¹ are attached to a nitrogen atom, optionally they join together with the nitrogen atom to form a 3 to 7-membered ring which optionally includes one or more other heteroatom selected from N, O and S, optionally the ring is substituted with one or more R¹ or R^{A}.

L is: 1) —C₁₋₆ alkyl, 2) —C₂₋₆ alkenyl, 3) —C₂₋₆ alkynyl, 4) —C₃₋₇ cycloalkyl, 5) —C₃₋₇ cycloalkenyl, 6) heterocyclyl, 7) —C₁₋₆ alkyl-C₃₋₇ cycloalkyl, 8) —C₁₋₆ alkyl-heterocyclyl, 9) aryl, or 10) heteroaryl. In this list, the alkyl, the alkenyl, the alkynyl, the cycloalkyl, the cycloalkenyl, the heterocyclyl, the aryl and the heteroaryl group are each independently optionally substituted with one or two R^{A} substituents.

R¹ is: 1) —H, 2) —C₁₋₆ alkyl, 3) —C₂₋₆ alkenyl, 4) —C₂₋₆ alkynyl, 5) —C₃₋₇ cycloalkyl, 6) —C₃₋₇cycloalkenyl, 7) —C₁₋₅ perfluorinated, 8) heterocyclyl, 9) aryl, 10) heteroaryl, 11) -benzyl, or 12) 5-[(3aS,4S,6aR)-2-oxohexahydro-1H-thieno[3,4-d]imidazole-4-yl]pentanoyl. In this list, the alkyl, the alkenyl, the alkynyl, the cycloalkenyl, the perfluorinated alkyl, the heterocyclyl, the aryl, the heteroaryl and the benzyl groups are each independently optionally substituted with 1, 2 or 3 R^{A} or R¹ substituents.

R² is: 1) —H, 2) —C₁₋₆ alkyl, 3) —SR¹, 4) —C(O)R¹, 5) —S(O)R¹, 6) —S(O)₂R¹, 7) -benzyl optionally substituted with 1, 2 or 3 R^{A} or R¹ substituents, 8) -L-heteroaryl optionally substituted with one or more R^{A} or R¹ substituents attached on either one or both the L and heteroaryl groups, 9) -L-heterocyclyl optionally substituted with one or more R^{A} or R¹ substituents attached on either one or both the L and heterocyclyl groups, 10) -L-aryl optionally substituted with one or more R^{A} or R¹ substituents attached on either one or both the L and the aryl groups, 11) -heteroaryl optionally substituted with one or more R^{A} or R¹ substituents, or 12) -aryl optionally substituted with one or more R^{A} or R¹ substituents. In this list, each substituent is optionally attached to the L group if it is not already present.

R^{A} is 1) -halogen, 2) —CF₃, 3) —OH, 4) —OR¹, 5) -L-OH, 6) -L-OR¹, 7) —OCF₃, 8) —SH, 9) —SR¹, 10) —CN, 11) —NO₂, 12) —NH₂, 13) NHR¹, 14) —N(R¹)R¹, 15) -L-NH₂, 16) -L-NHR¹, 17) -L-N(R¹)R¹, 18) -L-SR¹, 19) -L-S(O)R¹, 20) -L-S(O)₂R¹, 21) —C(O)OH, 22) —C(O)OR¹, 23) —C(O)NH₂, 24) —C(O)NHR¹, 25) —C(O)N(R¹)R¹, 26) —NHC(O)R¹, 27) —NR¹C(O)R¹, 28) —NHC(O)OR¹, 29) —NR¹C(O)OR¹, 30) —OC(O)NH₂, 31) —OC(O)NHR¹, 32) —OC(O)N(R¹)R¹, 33) —OC(O)R¹, 34) —C(O)R¹, 35) —NHC(O)NH₂, 36) —NHC(O)NHR¹, 37) —NHC(O)N(R¹)R¹, 38) —NR¹C(O)NH₂, 39) —NR¹C(O)NHR¹, 40) —NR¹C(O)N(R¹)R¹, 41) —NHS(O)₂R¹, 42) —NR¹S(O)₂R¹, 43) —S(O)₂NH₂, 44) —S(O)₂NHR¹, 45) —S(O)₂N(R¹)R¹, 46) —S(O)R¹, 47) —S(O)₂R¹, 48) —OS(O)₂R¹, 49) —S(O)₂OR¹, 50) -benzyl, 51) —N₃, or 52) —C(—N═N—)(CF₃). In this list, the benzyl group is optionally substituted with 1, 2 or 3 R^{A} or R¹ substituents.

m may be an integer from 1 to 6, wherein when m is 2 or more, Xᵢ are the same or different and are each independently NR¹, CH₂, O or S, wherein R¹ is as defined above, and Lᵢ are the same or different and are each independently L as defined above.

R³ and R⁴ may be the same or different and are each independently H, R¹ as defined above, or they join together with N to form a 3 to 7-membered ring which optionally includes one or more other heteroatom selected from N, O and S, optionally the ring is substituted with one or more R¹ or R^{A}.

In some embodiments, the pyrimido[4,5-b]indole compound may have the general formula IIA shown below

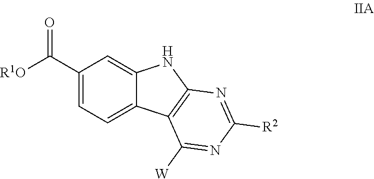

wherein R¹, W and R² are each defined above.

In some embodiments, the pyrimido[4,5-b]indole compound may have the general formula IIB shown below

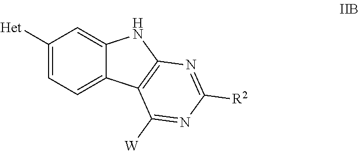

wherein W and R² are each defined above and Het is a 3 to 7-membered heterocycle, optionally substituted with one or more R¹ or R^{A} as defined above.

In some embodiments, the pyrimido[4,5-b]indole compound may have the general formula IIAA shown below

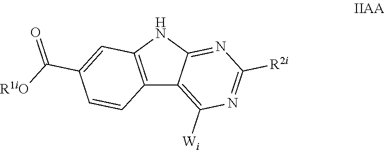

wherein R¹ⁱ is R¹ are each defined above.

In some embodiments, the pyrimido[4,5-b]indole compound may have the general formula IIBB shown below

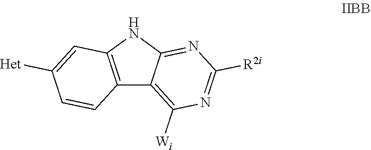

wherein Het is as defined above.

In the formulas IIAA and IIBB, the groups Wᵢ and R^{2′} may be independently selected from the group consisting of —H, —OH, —OR^{P}, —SH, —SR^{P}, —COOH, —COOR^{P}, —CONHR^{P}, —CONRR^{P}, —R^{P}, —NHC(O)R^{P}, —NR^{N}C(O)R^{P}, —NH₂, —NHR^{P}, —NR^{P}R^{Q}, halo, —CN, and —NO₂, where each —R^{N} is independently alkyl, each of —R^{P} and —R^{Q} is independently selected from alkyl, cycloalkyl, heterocyclyl, and aryl, or —R^{P} and —R^{Q} may together with the nitrogen to which they are attached form a heterocyclic group. The alkyl, cycloalkyl, heterocyclyl and aryl groups may each be optionally substituted.

In some embodiments, each of —R^{P} and —R^{Q} may be independently selected from C₁₋₆ alkyl, C₃₋₁₀ cycloalkyl, C₄₋₁₂ heterocyclyl and C₆₋₁₀ aryl. The alkyl, cycloalkyl, heterocyclyl and aryl groups may each be optionally substituted.

In some embodiments, each —R^{N} is C₁₋₆ alkyl.

In some embodiments, each —R^{N} is methyl.

Preferably, the substituent R²ⁱ in each of formula IIAA and IIBB is independently selected from H and —R^{P}, and more preferably wherein R^{P} is optionally substituted alkyl or aryl, such as optionally substituted alkyl, such as optionally substituted C₁₋₆ alkyl.

Preferably, the substituent W in each of formula IIAA and IIBB is independently selected from —NH₂, —NHR^{P}, —NR^{P}R^{Q} and —R^{P}, such as selected from —NHR^{P} and —NR^{P}R^{Q}.

In some embodiments, Wᵢ is —NHR^{P}.

In some embodiments, Wᵢ is 3-piperidin-1-ylpropylamino or 4-N-cyclohexane-1,4-diamine.

In some embodiments, R²ⁱ is selected from —H and benzyl.

In the formula IIAA, the substituent R¹ⁱ is preferably alkyl, such as C₁₋₆ alkyl, such as methyl. The alkyl group may be optionally substituted.

In the formula IIAA, the substituent R²ⁱ is preferably —H.

In the formula IIBB, the substituent R²ⁱ is preferably —R^{P}, and more preferably —R^{P} is optionally substituted alkyl or aryl, such as optionally substituted alkyl, such as optionally substituted C₁₋₆ alkyl.

In the formula IIBB, the substituent Het is preferably tetrazolyl, such as tetrazol-5-yl. The tetrazolyl group may be optionally substituted, for example substituted with methyl.

An optional substituent may be selected from cycloalkyl, heterocyclyl, aryl, —OH, —OR^{S}, —SH, —SR^{S}, —COOH, —COOR^{S}, —CONHR^{S}, —CONR^{S}R^{T}, —NHC(O)R^{S}, —NR^{N}C(O)R^{S}, —NH₂, —NHR^{S}, —NR^{S}R^{T}, halo, —CN, and —NO₂, where each —R^{N} is independently alkyl, each of —R^{S} and —R^{T} is independently selected from alkyl, cycloalkyl, heterocyclyl, and aryl, or —R^{S} and —R^{T} may together with the nitrogen to which they are attached form a heterocyclic group.

The optional substituents mentioned above are typically substituents to carbon. Where the group contains a nitrogen atom, for example a ring nitrogen atom in a heterocyclyl or aryl group, that nitrogen may be optionally substituted with alkyl, cycloalkyl, heterocyclyl, aryl, —COH, —COR^{S}, —CONHR^{S}, —CONR^{S}R^{T}, —NHC(O)R^{S}, and —NR^{N}C(O)R^{S}, where —R^{N}, —R^{S} and —R^{T} are as defined above.

As used herein, the term “alkyl” is intended to include both branched and straight chain saturated aliphatic hydrocarbon groups having the specified number of carbon atoms, for example, C₁₋₆ in C₁₋₆ alkyl is defined as including groups having 1, 2, 3, 4, 5 or 6 carbons in a linear or branched saturated arrangement. Examples of C₁₋₆ alkyl as defined above include, but are not limited to, methyl, ethyl, n-propyl, i-propyl, n-butyl, t-butyl, i-butyl, pentyl and hexyl. An alkyl group may be optionally substituted.

As used herein, the term “cycloalkyl” is intended to mean a monocyclic saturated aliphatic hydrocarbon group having the specified number of carbon atoms, for example, C₃-7 in C₃₋₇ cycloalkyl is defined as including groups having 3, 4, 5, 6 or 7 carbons in a monocyclic saturated arrangement. Examples of C₃₋₇ cycloalkyl as defined above include, but are not limited to, cyclopropyl, cyclobutyl, cyclopentyl, cyclohexyl and cycloheptyl. A cycloalkyl group may be optionally substituted.

As used herein, the term “alkenyl” is intended to mean unsaturated straight or branched chain hydrocarbon groups having the specified number of carbon atoms therein, and in which at least two of the carbon atoms are bonded to each other by a double bond, and having either E or Z regiochemistry and combinations thereof. For example, C₂₋₆ in C₂₋₆ alkenyl is defined as including groups having 2, 3, 4, 5 or 6 carbons in a linear or branched arrangement, at least two of the carbon atoms being bonded together by a double bond. Examples of C₂₋₆ alkenyl as defined above include, but are not limited to, ethenyl (vinyl), 1-propenyl, 2-propenyl, 1-butenyl and the like.

As used herein, the term “alkynyl” is intended to mean unsaturated, straight chain hydrocarbon groups having the specified number of carbon atoms therein, and in which at least two carbon atoms are bonded together by a triple bond. For example, C₂₋₄ alkynyl is defined as including groups having 2, 3 or 4 carbon atoms in a chain, at least two of the carbon atoms being bonded together by a triple bond. Examples of such alkynyl include, but are not limited to, ethynyl, 1-propynyl, 2-propynyl and the like.

As used herein, the term “cycloalkenyl” is intended to mean a monocyclic unsaturated hydrocarbon group having the specified number of carbon atoms therein, and in which at least two of the carbon atoms are bonded to each other by a double bond. For example, C₃₋₇ in C₃₋₇ cycloalkenyl is defined as including groups having 3, 4, 5, 6 or 7 carbons in a monocyclic arrangement, at least two of the carbon atoms being bonded together by a double bond. Examples of C₃₋₇ cycloalkenyl as defined above include, but are not limited to, cyclopentenyl, cyclohexenyl and the like.

As used herein, the term “halo” or “halogen” is intended to mean fluorine, chlorine, bromine or iodine.

As used herein, the term “aryl”, either alone or in combination with another radical, means a carbocyclic aromatic monocyclic group containing 6 carbon atoms which may be further fused to a second 5- or 6-membered carbocyclic group which may be aromatic, saturated or unsaturated. Examples of aryl include, but are not limited to, phenyl, indanyl, 1-naphthyl, 2-naphthyl, tetrahydronaphthyl and the like. The aryl may be connected to another group either at a suitable position on the cycloalkyl ring or the aromatic ring. An aryl group may be optionally substituted.

As used herein, the term “heteroaryl” is intended to mean a monocyclic or bicyclic ring system of up to 10 atoms, wherein at least one ring is aromatic, and contains from 1 to 4 hetero atoms selected from the group consisting of O, N, and S. The heteroaryl may be attached either via a ring carbon atom or one of the heteroatoms. Examples of heteroaryl include, but are not limited to, thienyl, benzimidazolyl, benzo[b]thienyl, furyl, benzofuranyl, pyranyl, isobenzofuranyl, chromenyl, xanthenyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinoxalinyl, quinazolinyl, cinnolinyl, pteridinyl, isothiazolyl, isochromanyl, chromanyl, isoxazolyl, furazanyl, indolinyl, isoindolinyl, thiazolo[4,5-b]-pyridine, tetrazolyl, oxadiazolyl, thiadiazolyl, thienyl and fluorescein derivatives. A heteroaryl group may be optionally substituted.

As used herein, the term “heterocycle,” “heterocyclic” or “heterocyclyl” is intended to mean a 3, 4, 5, 6, or 7 membered non-aromatic ring system containing from 1 to 4 heteroatoms selected from the group consisting of O, N and S. Examples of heterocycles include, but are not limited to, pyrrolidinyl, tetrahydrofuranyl, piperidyl, 3,5-dimethylpiperidyl, pyrrolinyl, piperazinyl, imidazolidinyl, morpholinyl, imidazolinyl, pyrazolidinyl, pyrazolinyl, and the like, where the attachment to the ring can be on either the nitrogen atom or a carbon atom of the ring. A heterocyclyl group may be optionally substituted.

As used herein, the term “optionally substituted with one or more substituents” or its equivalent term “optionally substituted with at least one substituent” is intended to mean that the subsequently described event of circumstances may or may not occur, and that the description includes instances where the event or circumstance occurs and instances in which it does not. The definition is intended to mean from zero to five substituents.

In some preferred embodiments, the pyrimidoindole compound is methyl 4-(3-piperidin-1-ylpropylamino)-9H-pyrimido[4,5-b]indole-7-carboxylate (CAS 1448723-60-1; UM729):

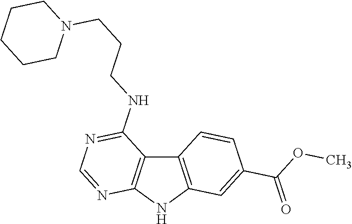 [SMILES: COC(=O)c1ccc2c(c1)[nH]c1ncnc(NCCCN3CCCCC3)c12]

In other embodiments, the pyrimidoindole compound is (1R,4R)—N1-(2-benzyl-7-(2-methyl-2H-tetrazol-5-yl)-9H-pyrimido[4,5-b]indol-4-yl)cyclohexane-1,4-diamine (CAS: 1448724-09-1; UM171):

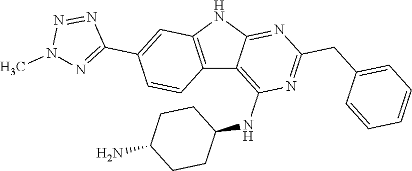 [SMILES: Cn1nnc(-c2ccc3c(c2)[nH]c2nc(Cc4ccccc4)nc(N[C@H]4CC[C@H](N)CC4)c23)n1]

The preparation of pyrimidoindole compounds is described in U.S. Ser. No. 10/336,747B32 and WO2013110198, the contents of which are hereby incorporated by reference in their entirety for all purposes.

The compounds may be provided and used as salt, prodrug, solvate and crystal forms thereof.

Examples of salts of the compounds include all pharmaceutically acceptable salts, such as, without limitation, acid addition salts of strong mineral acids such as HCl and HBr salts and addition salts of strong organic acids such as a methanesulfonic acid salt. Further examples of salts include sulfates and acetates such as acetate itself, trifluoroacetate or trichloroacetate.

Examples of solvates include hydrates, such as monohydrate.

Haematopoletic progenitor cells (HPCs) may be differentiated into progenitor T cells as described herein in the absence of stromal cells, such as OP9-DI4 stromal cells, feeder cells or serum.

A lymphoid expansion medium (also referred to herein as lymphoid progenitor medium or stage 4 medium) is a cell culture medium that promotes the lymphoid differentiation of HPCs into progenitor T cells.

A suitable lymphoid expansion medium may (i) stimulate cKIT receptor (CD117; KIT receptor tyrosine kinase) and/or cKIT receptor (CD117: KIT receptor tvrosine kinase) mediated sianallina pathwavs. (ii) stimulate MPL (CD110) and/or mediated signalling pathways (iii) FLT3 and/or FLT3 mediated signalling pathways and (iv) have interleukin (IL) activity. For example, a lymphoid expansion medium may comprise the differentiation factors SCF, FLT3L, TPO and IL-7.

In preferred embodiments, the lymphoid expansion medium is a chemically defined medium. For example, the lymphoid expansion medium may consist of a chemically defined nutrient medium supplemented with effective amounts of the above differentiation factors. Suitable lymphoid expansion media are well-known in the art and include Stemspan™ SFEM II (Cat #9605; StemCell Technologies Inc, CA). with Stemspan™ lymphoid expansion supplement (Cat #9915; StemCell Technologies Inc, CA).

The lymphoid expansion medium may be supplemented with an amount of the pyrimidoindole compound that is effective to increase the proportion of progenitor T cells in the differentiated population and/or increase the expansion of the progenitor T cell population. For example, the medium may be supplemented with a concentration of 0.1 μM or greater, 0.25 μM or greater, 0.5 μM or greater or 1 μM or greater pyrimidoindole compound.

In some embodiments, the lymphoid expansion medium may be supplemented with an amount of the pyrimidoindole compound that is insufficient to exert cytotoxic effects on the cells cultured therein. For example, the medium may be supplemented with a concentration of less than 10 μM, less than 7.5 μM, less than 5 μM, less than 4 μM, less than 3 μM, or less than 2 μM. For example, the medium may be supplemented with a concentration of 1 μM or less pyrimidoindole compound. In some preferred embodiments, the lymphoid expansion medium may be supplemented with 0.1 μM to 10 μM, 0.1 μM to 5 μM or 0.1 μM to 2 μM of the pyrimidoindole compound, preferably 0.25 μM to 5 μM or 0.25 μM to 21 μM of the pyrimidoindole compound.

For example, the lymphoid expansion medium may be supplemented with UM729 at a concentration of less than 5 μM, less than 4 μM, less than 3 μM, or less than 2 μM, for example a concentration of 1p M or less. In some preferred embodiments, the lymphoid expansion medium may be supplemented with 0.1 μM to 2 μM preferably 0.25 μM to 2 μM UM729. The lymphoid expansion medium may be supplemented with UM171 at a concentration of less than 1 μM or less than 0.5 μM, for example a concentration of 0.25 μM or less. In some preferred embodiments, the lymphoid expansion medium may be supplemented with a concentration of 0.01 μM to 0.25 μM UM171, preferably 0.04 μM to 0.25 μM UM171.

For example, the progenitor T cells, e.g., those at stage 4 (CD7+, CD5+), can be expanded in the presence of a pyrimidoindole compound (e.g., as supplement to the lymphoid expansion medium) for at least 7 days. In some embodiments, the expansion of the progenitor T cells at stage 4 is for at least 14 days, 21 days, 28 days, 35 days, 42 days, 49 days, 56 days or 63 days.

In some embodiments, in subsequent differentiation stages following the expansion, the cells are cultured in the absence of the pyrimidoindole compound (for example the culture media may not be supplemented with pyrimidoindole compound. In some embodiments, the cells are cultured in stage 5 the absence of the pyrimidoindole compound.

The HPCs may be cultured on a surface during differentiation into progenitor T cells. For example, the HPCs may be cultured on a surface of a culture vessel, bead or other biomaterial or polymer.

Preferably, the surface may be coated with a factor that stimulates Notch signalling, for example a Notch ligand, such as Delta-like 1 (DLL1) or Delta-like 4 (DLL4). Suitable Notch ligands are well-known in the art and available from commercial suppliers.

The surface may also be coated with an extracellular matrix protein, such as fibronectin, vitronectin, laminin or collagen and/or one or more cell surface adhesion proteins, such as VCAM1. In some embodiments, the surface for HPC culture may have a coating that comprises a factor that stimulates Notch signalling, for example a Notch ligand, such as DLL4, without the extracellular matrix protein or cell surface adhesion protein.

In some embodiments, the surface for HPC culture may have a coating that comprises a factor that stimulates Notch signalling, for example a Notch ligand, such as DLL4, an extracellular matrix protein, such as vitronectin, and a cell surface adhesion protein, such as VCAM1. The surface may be coated with an extracellular matrix protein, factor that stimulates Notch signalling and cell surface adhesion protein by contacting the surface with a coating solution. For example, the coating solution may be incubated on the surface under suitable conditions to coat the surface. Conditions may, for example, include about 2 hours at room temperature. Coating solutions comprising an extracellular matrix protein and a factor that stimulates Notch signalling are available from commercial suppliers (StemSpan™ Lymphoid Differentiation Coating Material; Cat #9925; Stem Cell Technologies Inc, CA).

The HPCs may be cultured in the lymphoid expansion medium on the substrate for a time sufficient for the HPCs to differentiate into progenitor T cells. For example, the HPCs may be cultured for 2-6 weeks, 2 to 5 weeks or 2-4 weeks, preferably 3 weeks. In some embodiments, the HPCs may be cultured in the lymphoid expansion medium for 21 to 26 days. For example, the HPCs may be cultured from day 16 to any one of days 37 to 42 of the differentiation protocol in the lymphoid expansion medium.

A population of cells produced by culturing HPCs in the lymphoid expansion medium in the presence of a pyrimidoindole compound as described herein may have an increased proportion of progenitor T cells, for example T cells that express CD5 and CD7, relative to populations produced in the absence of pyrimidoindole compound. For example, the proportion of progenitor T cells in a population produced using pyrimidoindole compound may be at least 10%, at least 20%, at least 30%, at least 40%, at least 50%, at least 60%, at least 70%, at least 90%, at least 95% or at least 100% higher than the proportion of progenitor T cells in a population produced without pyrimidoindole compound.

In some embodiments, the amount or proportion of CD45+CD34+CD7+CD5-lymphoid cells (CLPs) in the population may also be increased by culturing HPCs in the lymphoid expansion medium in the presence of a pyrimidoindole compound, such as UM171. A population of cells produced by culturing HPCs in the lymphoid expansion medium in the presence of a pyrimidoindole compound, such as UM171, may have an increased proportion of CD45+CD34+CD7+CD5− lymphoid cells (CLPs) relative to populations produced in the absence of the pyrimidoindole compound. The proportion of CLPs in a population produced using the pyrimidoindole compound may be at least 10%, at least 20%, at least 30%, at least 40%, at least 50%, at least 60%, at least 70%, at least 90%, at least 90% or at least 100% higher than the proportion of CLPs in a population produced without the pyrimidoindole compound.

A method described herein may further comprise providing a population of haematopoietic progenitor cells (HPCs) for use in a method of producing progenitor T cells as described herein. In some preferred embodiments, HPCs are produced in vitro from induced pluripotent stem cells (iPSCs).

For example, a population of progenitor T cells may be produced by a method comprising;
(i) differentiating a population of iPSCs into HPCs, and
(ii) differentiating the HPCs into T cell progenitors in presence of a pyrimidoindole compound.

Optionally, method may further comprise (iii) expanding the population of progenitor T cells by culturing the population in presence of a pyrimidoindole compound.

iPSCs may be differentiated into HPCs using a three-step process that includes mesoderm and haemogenic endothelium stages. For example, a method may comprise;
(i) differentiating the population of iPSCs into mesoderm cells,
(ii) differentiating the mesoderm cells into haemogenic endothelial cells, and
(iii) differentiating the haemogenic endothelial cells into a population of HPCs.

Induced pluripotent stem cells (iPSCs) are pluripotent cells which are derived from non-pluripotent, fully differentiated donor or antecedent cells. iPSCs are capable of self-renewal in vitro and exhibit an undifferentiated phenotype and are potentially capable of differentiating into any foetal or adult cell type of any of the three germ layers (endoderm, mesoderm and ectoderm). The population of iPSCs may be clonal i.e. genetically identical cells descended from a single common ancestor cell. iPSCs may express one or more of the following pluripotency associated markers: POU5f1 (Oct4), Sox2, Alkaline Phosphatase, SSEA-3, Nanog, SSEA-4, Tra-1-60, KLF4 and c-myc, preferably one or more of POU5f1, NANOG and SOX2. An iPSC may lack markers associated with specific differentiative fates, such as Bra, Sox17, FoxA2, aFP, Sox1, NCAM, GATA6, GATA4, Hand1 and CDX2. In particular, an iPSC may lack markers associated with endodermal fates.

Preferably, the iPSCs are human IPSCs (hiPSCs).

In some embodiments, iPSCs may be gene edited, for example to inactivate or delete HLA genes or other genes associated with immunogenicity or GVHD or may be gene edited to include nucleic acid encoding an exogenous antigen receptor such as for example a TCR, CAR or NKCR.

IPSCs may be derived or reprogramed from donor cells, which may be somatic cells or other antecedent cells obtained from a source, such as a donor individual. The donor cells may be mammalian, preferably human cells. Suitable donor cells include adult fibroblasts and blood cells, for example peripheral blood cells, such as HPCs or mononuclear cells.

Suitable donor cells for reprogramming into iPSCs as described herein may be obtained from a donor individual. In some embodiments, the donor individual may be the same person as the recipient individual to whom the T cells will be administered following production as described herein (autologous treatment). In other embodiments, the donor individual may be a different person to the recipient individual to whom the T cells will be administered following production as described herein (allogeneic treatment). For example, the donor individual may be a healthy individual who is human leukocyte antigen (HLA) matched (either before or after donation) with a recipient individual suffering from cancer. In other embodiments, the donor individual may not be HLA matched with the recipient individual. Preferably, the donor individual may be a neonate (new-born), for example the donor cells may be obtained from a sample of umbilical cord blood.

Suitable donor individuals are preferably free of communicable viral (e.g. HIV, HPV, CMV) and adventitious agents (e.g. bacteria, mycoplasma), and free of known genetic abnormalities.

In some embodiments, a population of peripheral blood cells, such as HPCs, for reprogramming may be isolated from a blood sample, preferably an umbilical cord sample, obtained from the donor individual. Suitable methods for the isolation of HPCs and other peripheral blood cells, are well-known in the art and include, for example magnetic activated cell sorting (see, for example, Gaudernack et al., J Immunol Methods 1986 90 179), fluorescent activated cell sorting (FACS: see for example, Rheinherz et al., PNAS 1979 76 4061), and cell panning (see for example, Lum et al., Cell Immunol 1982 72 122). HPCs may be identified in a sample of blood cells by expression of CD34. In other embodiments, a population of fibroblasts for reprogramming may be isolated from a skin biopsy following dispersal using collagenase or trypsin and out-growth in appropriate cell culture conditions.

In some embodiments, IPSCs may be derived from antigen-specific T cells. For example, the T cells may comprise nucleic acid encoding αβ TCRs that bind to an antigen, such as a tumor antigen, displayed in complex with a class 1 MHC. Antigen-specific T cells for use in the generation of iPSCs may be obtained by screening a diverse population of T cells with peptide epitopes from the target antigen displayed on a class I or II MHC molecule on the surface of an antigen presenting cell, such as a dendritic cell, or by isolating from a tumour sample from a cancer patient.

Donor cells are typically reprogrammed into iPSCs by the introduction of reprogramming factors, such as Oct4, Sox2 and Klf4 into the cell. The reprogramming factors may be proteins or encoding nucleic acids and may be introduced into the differentiated cells by any suitable technique, including plasmid, transposon or more preferably, viral transfection or direct protein delivery. Other reprogramming factors, for example Klf genes, such as Klf-1, -2, -4 and -5; Myc genes such as C-myc, L-myc and N-myc; Nanog; SV40 Large T antigen; Lin28; and short hairpins (shRNA) targeting genes such as p53, may also be introduced into the cell to increase induction efficiency. Following introduction of the reprogramming factors, the donor cells may be cultured. Cells expressing pluripotency markers may be isolated and/or purified to produce a population of iPSCs. Techniques for the production of iPSCs are well-known in the art (Yamanaka et al., Nature 2007; 448:313-7; Yamanaka 6 2007 Jun. 7; 1(1):39-49; Kim et al., Nature. 2008 Jul. 31; 454(7204):646-50; Takahashi Cell. 2007 Nov. 30; 131(5):861-72. Park et al., Nature. 2008 Jan. 10; 451(7175):141-6; Kimet et al., Cell Stem Cell. 2009 Jun. 5; 4(6):472-6; Vallier, L., et al. Stem Cells, 2009. 9999(999A): p. N/A; Baghbaderani et al., 2016; Stem Cell Rev. 2016 August; 12(4):394-420; Baghbaderani et al. (2015) Stem Cell Reports, 5(4), 647-659).

Conventional techniques may be employed for the culture and maintenance of iPSCs (Vallier, L. et al., Dev. Biol. 275, 403-421 (2004), Cowan, C. A. et al. N. Engl. J. Med. 350, 1353-1356 (2004), Joannides, A. et al. Stem Cells 24, 230-235 (2006) Klimanskaya, I. et al. Lancet 365, 1636-1641 (2005), Ludwig, T. E. et al. Nat. Biotechnol. 24, 185-187 (2006)). IPSCs for use in the present methods may be grown in defined conditions or on feeder cells. For example, iPSCs may be conventionally cultured in a culture dish on a layer of feeder cells, such as irradiated mouse embryonic fibroblasts (MEF), at an appropriate density (e.g. 10⁵ to 10⁶ cells/60 mm dish), or on an appropriate substrate, in a feeder conditioned or defined iPSC maintenance medium. iPSCs for use in the present methods may be passaged by enzymatic or mechanical means. In some embodiments, iPSCs may be passaged on Matrigel™ or an ECM protein, such as vibronectin, in an iPSC maintenance medium, such as mTeSR™1 or TeSR™2 (StemCell Technologies) or E8 flex (Life Thermo) culture medium.

Differentiation and maturation of the cell populations in the steps of the methods described herein is induced by culturing the cells in a culture medium supplemented with a set of differentiation factors. The set of differentiation factors that is listed for each culture medium is preferably exhaustive and medium may be devoid of other differentiation factors. In preferred embodiments, the culture media are chemically defined media. For example, a culture medium may consist of a chemically defined nutrient medium that is supplemented with an effective amount of one or more differentiation factors, as described below. A chemically defined nutrient medium may comprise a basal medium that is supplemented with one or more serum-free culture medium supplements.

Differentiation factors are factors which modulate, for example promote or inhibit, a signalling pathway which mediates differentiation in a mammalian cell. Differentiation factors may include growth factors, cytokines and small molecules which modulate one or more of the Activin/Nodal, FGF, Wnt or BMP or signalling pathways thereof. Examples of differentiation factors include Activin/Nodal, FGFs, BMPs, retinoic acid, vascular endothelial growth factor (VEGF), stem cell factor (SCF), TGFβ ligands, GDFs, LIF, Interleukins, GSK-3 inhibitors and phosphatidylinositol 3-kinase (PI3K) inhibitors.

Differentiation factors which are used in one or more of the media described herein include TGFβ ligands, such as activin, fibroblast growth factor (FGF), bone morphogenetic protein (BMP), stem cell factor (SCF), vascular endothelial growth factor (VEGF), GSK-3 inhibitors (such as CHIR-99021), interleukins, and hormones, such as IGF-1 and angiotensin II. A differentiation factor may be present in a medium described herein in an amount that is effective to modulate a signalling pathway in cells cultured in the medium.

In some embodiments, a differentiation factor listed above or below may be replaced in a culture medium by a factor that has the same effect (i.e. stimulation or inhibition) on the same signalling pathway. Suitable factors are known in the art and include proteins, nucleic acids, antibodies and small molecules.

The extent of differentiation of the cell population during each step may be determined by monitoring and/or detecting the expression of one or more cell markers in the population of differentiating cells. For example, an increase in the expression of markers characteristic of the more differentiated cell type or a decrease in the expression of markers characteristic of the less differentiated cell type may be determined. The expression of cell markers may be determined by any suitable technique, including immunocytochemistry, immunofluorescence, R^{T}-PCR, immunoblotting, fluorescence activated cell sorting (FACS), and enzymatic analysis. In preferred embodiments, a cell may be said to express a marker if the marker is detectable on the cell surface. For example, a cell which is stated herein not to express a marker may display active transcription and intracellular expression of the marker gene but detectable levels of the marker may not be present on the surface of the cell.

A population of partially differentiated cells, for example mesoderm cells, haemogenic endothelium (HE; i.e. haemogenic endothelial cells or HECs), HPCs, or T cell progenitors, that is produced by a step in the methods described herein may be cultured, maintained or expanded before the next differentiation step. Partially differentiated cells may be expanded by any convenient technique.

After each step, the population of partially differentiated cells which is produced by that step may be free or substantially free from other cell types. For example, the population may contain 60% or more, 70% or more, 80% or more or 90% or more partially differentiated cells, following culture in the medium. Preferably, the population of cells is sufficiently free of other cell types that no purification is required. If required, the population of partially differentiated cells may be purified by any convenient technique, such as MACs or FACS.

Cells may be cultured in a monolayer, in the absence of feeder cells, on a surface or substrate coated with extracellular matrix protein, such as fibronectin, laminin or collagen. Suitable techniques for cell culture are well-known in the art (see, for example, Basic Cell Culture Protocols, C. Helgason, Humana Press Inc. U.S. (15 Oct. 2004) ISBN: 1588295451; Human Cell Culture Protocols (Methods in Molecular Medicine S.) Humana Press Inc., U.S. (9 Dec. 2004) ISBN: 1588292223; Culture of Animal Cells: A Manual of Basic Technique, R. Freshney, John Wiley & Sons Inc (2 Aug. 2005) ISBN: 0471453293, Ho W Y et al., J Immunol Methods. (2006) 310:40-52, Handbook of Stem Cells (ed. R. Lanza) ISBN: 0124366430) Basic Cell Culture Protocols' by J. Pollard and J. M. Walker (1997), ‘Mammalian Cell Culture: Essential Techniques’ by A. Doyle and J. B. Griffiths (1997), ‘Human Embryonic Stem Cells’ by A. Chiu and M. Rao (2003), Stem Cells: From Bench to Bedside' by A. Bongso (2005), Peterson & Loring (2012) Human Stem Cell Manual: A Laboratory Guide Academic Press and ‘Human Embryonic Stem Cell Protocols’ by K. Turksen (2006). Media and ingredients thereof may be obtained from commercial sources (e.g. Gibco, Roche, Sigma, Europa bioproducts, R&D Systems). Standard mammalian cell culture conditions may be employed for the above culture steps, for example 37° C., 5% or 21% Oxygen, 5% Carbon Dioxide. Media is preferably changed every two days and cells allowed to settle by gravity.

Cells may be cultured in a culture vessel. Suitable cell culture vessels are well-known in the art and include culture plates, dishes, flasks, bioreactors, and multi-well plates, for example 6-well, 12-well or 96-well plates. The culture vessel is preferably treated for tissue culture, for example by coating one or more surfaces of the vessel with an extracellular matrix protein, such as fibronectin, laminin or collagen. Culture vessels may be treated for tissue culture using standard techniques, for example by incubating with a coating solution as described herein or may be obtained from pre-treated from commercial suppliers.

In a first stage, iPSCs may be differentiated into mesoderm cells by culturing the population of iPSCs under suitable conditions to promote mesodermal differentiation. For example, the iPSCs cells may be cultured sequentially in first, second and third mesoderm induction media to induce differentiation into mesoderm cells.

A suitable first mesoderm induction medium may stimulate SMAD2 and SMAD3 and/or SMAD2 and SMAD3 mediated signalling pathways. For example, the first mesoderm induction medium may comprise activin.

A suitable second mesoderm induction medium may (i) stimulate SMAD1, SMAD2, SMAD3, SMAD5 and SMAD9 and/or SMAD1, SMAD2, SMAD3, SMAD5 and SMAD9 mediated signalling pathways and (ii) have fibroblast growth factor (FGF) activity. For example, the second mesoderm induction medium may comprise activin, preferably activin A, BMP, preferably BMP4 and FGF, preferably bFGF.

A suitable third mesoderm induction medium may (i) stimulate SMAD1, SMAD2, SMAD3, SMAD5 and SMAD9 and/or SMAD1, SMAD2, SMAD3, SMAD5 and SMAD9 mediated signalling pathways (ii) have fibroblast growth factor (FGF) activity and (iii) inhibit glycogen synthase kinase 33. For example, the third mesoderm induction medium may comprise activin, preferably activin A, BMP, preferably BMP4, FGF, preferably bFGF, and a GSK3 inhibitor, preferably CHIR99021.

The first, second and third mesoderm induction media may be devoid of differentiation factors other than the differentiation factors set out above.

SMAD2 and SMAD3 and/or SMAD2 and SMAD3 mediated intracellular signalling pathways may be stimulated by the first, second and third mesoderm induction media through the presence in the media of a first TGFβ ligand. The first TGFβ ligand may be Activin. Activin (Activin A: NCBI Gene ID: 3624 nucleic acid reference sequence NM_002192.2 GI: 62953137, amino acid reference sequence NP_002183.1 GI: 4504699) is a dimeric polypeptide which exerts a range of cellular effects via stimulation of the Activin/Nodal pathway (Vallier et al., Cell Science 118:4495-4509 (2005)). Activin is readily available from commercial sources (e.g. Stemgent Inc. MA USA; Miltenyi Biotec Gmbh, DE). Conveniently, the concentration of Activin in a medium described herein may be from 1 to 100 ng/ml, preferably about 5 to 50 ng/ml.

The fibroblast growth factor (FGF) activity of the second and third mesoderm induction media may be provided by the presence of fibroblast growth factor (FGF) in the media. Fibroblast growth factor (FGF) is a protein factor which stimulates cellular growth, proliferation and cellular differentiation by binding to a fibroblast growth factor receptor (FGFR). Suitable fibroblast growth factors include any member of the FGF family, for example any one of FGF1 to FGF14 and FGF15 to FGF23. Preferably, the FGF is FGF2 (also known as bFGF, NCBI GenelD: 2247, nucleic acid sequence NM_002006.3 GI: 41352694, amino acid sequence NP_001997.4 GI: 41352695); FGF7 (also known as keratinocyte growth factor (or KGF), NCBI GenelD: 2247, nucleic acid sequence NM_002006.3 GI: 41352694, amino acid sequence NP_001997.4 GI: 41352695); or FGF10 (NCBI GenelD: 2247, nucleic acid sequence NM_002006.3 GI: 41352694, amino acid sequence NP_001997.4 GI: 41352695). Most preferably, the fibroblast growth factor is FGF2.

Conveniently, the concentration of FGF, such as FGF2 in a medium described herein may be from 0.5 to 50 ng/ml, preferably about 5 ng/ml. Fibroblast growth factors, such as FGF2, FGF7 and FGF10, may be produced using routine recombinant techniques or obtained from commercial suppliers (e.g. R&D Systems, Minneapolis, MN; Stemgent Inc, USA; Miltenyi Biotec Gmbh, DE).

SMAD1, SMAD5 and SMAD9 and/or SMAD1, SMAD5 and SMAD9 mediated intracellular signalling pathways may be stimulated by the second and third mesoderm induction media through the presence in the media of a second TGFβ ligand.

The second TGFβ ligand may be a Bone Morphogenic Protein (BMP). Bone Morphogenic Proteins (BMPs) bind to Bone Morphogenic Protein Receptors (BMPRs) and stimulate intracellular signalling through pathways mediated by SMAD1, SMAD5 and SMAD9. Suitable Bone Morphogenic Proteins include any member of the BMP family, for example BMP2, BMP3, BMP4, BMP5, BMP6 or BMP7. Preferably the second TGFβ ligand is BMP2 (NCBI GenelD: 650, nucleic acid sequence NM_001200.2 GI: 80861484; amino acid sequence NP_001191.1 GI: 4557369) or BMP4 (NCBI GenelD: 652, nucleic acid sequence NM_001202.3 GI: 157276592; amino acid sequence NP_001193.2 GI: 157276593). Suitable BMPs include BMP4. Conveniently, the concentration of a Bone Morphogenic Protein, such as BMP2 or BMP4 in a medium described herein may be from 1 to 500 ng/ml, preferably about 10 ng/ml. BMPs may be produced using routine recombinant techniques or obtained from commercial suppliers (e.g. R&D, Minneapolis, USA, Stemgent Inc, USA; Miltenyi Biotec Gmbh, DE).

The GSK3p inhibition activity of the third mesoderm induction medium may be provided by the presence of a GSK33 inhibitor in the medium. GSK33 inhibitors inhibit the activity of glycogen synthase kinase 33 (Gene ID 2932: EC2.7.11.26). Preferred inhibitors specifically inhibit the activity of glycogen synthase kinase 33.

Suitable inhibitors include CHIR99021 (6-((2-((4-(2,4-Dichlorophenyl)-5-(4-methyl-1H-imidazol-2-yl)pyrimidin-2-yl)amino)ethyl)amino)nicotinonitrile; Ring D. B. et al., Diabetes, 52:588-595 (2003)) alsterpaullone, kenpaullone, BIO(6-bromoindirubin-3′-oxime (Sato et al., Nat Med. 2004 January; 10(1):55-63), SB216763 (3-(2, 4-dichlorophenyl)-4-(1-methyl-1H-indol-3-yl)-1H-pyrrole-2,5-dione), Lithium and SB415286 (3-[(3-chloro-4-hydroxyphenyl)amino]-4-(2-nitrophenyl)-1H-pyrrole-2,5-dione; Coghlan et al., Chem Biol. 2000 October; 7(10):793-803). In some preferred embodiments, the GSK3p inhibitor is CHIR99021. Suitable glycogen synthase kinase 33 inhibitors may be obtained from commercial suppliers (e.g. Stemgent Inc. MA USA; Cayman Chemical Co. MI USA; Selleckchem, MA USA). For example, the third mesoderm induction medium may contain 0.1 to 100 μM of a GSK33 inhibitor, such as CHIR99021, preferably about 10 μM.

In preferred embodiments, the first, second and third mesoderm induction media are chemically defined media. For example, the first mesoderm induction medium may consist of a chemically defined nutrient medium supplemented with an effective amount of activin, preferably activin A, for example 50 ng/ml activin A; the second mesoderm induction medium may consist of a chemically defined nutrient medium supplemented with an effective amount of activin preferably activin A, for example 5 ng/ml activin A, BMP, preferably BMP4, for example 10 ng/ml BMP4; and FGF, preferably bFGF (FGF2), for example 5 ng/ml bFGF; and the third mesoderm induction medium may consist of a chemically defined nutrient medium supplemented with an effective amount of activin preferably activin A, for example 5 ng/ml activin A, BMP, preferably BMP4, for example 10 ng/ml BMP4; FGF, preferably bFGF (FGF2), for example 5 ng/ml bFGF; and GSK3 inhibitor, preferably CHIR-99021, for example 10 μM CHIR-99021.

A chemically defined medium (CDM) is a nutritive solution for culturing cells which contains only specified components, preferably components of known chemical structure. A CDM is devoid of undefined components or constituents which include undefined components, such as feeder cells, stromal cells, serum, serum albumin and complex extracellular matrices, such as Matrigel™ For example, a CDM does not contain stromal cells, such as OP9 cells, expressing Notch ligands, such as DLL1 or DLL4.

The CDM or chemically defined nutrient medium may comprise a chemically defined basal medium. Suitable chemically defined basal media include Iscove's Modified Dulbecco's Medium (IMDM), Ham's F12, Advanced Dulbecco's modified eagle medium (DMEM) (Price et al., Focus (2003), 25 3-6), Williams E (Williams, G. M. et al., Exp. Cell Research, 89, 139-142 (1974)), RPMI-1640 (Moore, G. E. and Woods L. K., (1976) Tissue Culture Association Manual. 3, 503-508) and StemPro™-34 (ThermoFisher Scientific).

The basal medium may be supplemented by serum-free culture medium supplements and/or additional components in the medium. Suitable supplements and additional components are described above and may include L-glutamine or substitutes, such as GlutaMAX-1™, ascorbic acid, monothiolglycerol (MTG), antibiotics such as penicillin and streptomycin, human serum albumin, for example recombinant human serum albumin, such as Cellastim™ (Merck/Sigma) and Recombumin™ (albumedix.com), insulin, transferrin and 2-mercaptoethanol. A basal medium may be supplemented with a serum substitute, such as Knockout Serum Replacement (KOSR; Invitrogen).

The iPSCs may be cultured in the first mesoderm induction medium for 1 to 12 hours, for example any of 1, 2, 3, 4, 5, 6, 7, 8, 9, 10, 11 or 12 hours, preferably about 4 hours; then cultured in the second mesoderm induction medium for 30 to 54 hours, for example any of 35, 36, 37, 38, 39, 40, 41, 42, 43, 44, 45, 46, 47, 48, 49 or 50 hours, preferably about 44 hours; and then cultured in the third mesoderm induction medium for 36 to 60 hours, for example any of 37, 38, 39, 40, 41, 42, 43, 44, 45, 46, 47, 48, 49, 50, 51, 52 or 53 hours, preferably about 48 hours to produce a population of mesodermal cells.

Mesoderm cells are partially differentiated progenitor cells that are committed to mesodermal lineages and are capable of differentiation under appropriate conditions into all cell types in the mesenchyme (fibroblast), muscle, bone, adipose, vascular and haematopoietic systems. Mesoderm cells may express one or more mesodermal markers. For example, the mesoderm cells may express any one, two, three, four, five, six or all seven of Brachyury, Goosecoid, Mixl1, KDR, FoxA2, GATA6 and PDGFaR.

In a second stage, mesoderm cells may be differentiated into haemogenic endothelial (HE) cells by culturing the population of mesoderm cells under suitable conditions to promote HE differentiation. For example, the mesoderm cells may be cultured in an HE induction medium.

A suitable HE induction medium may (i) stimulate cKIT receptor (CD117; KIT receptor tyrosine kinase) and/or cKIT receptor (CD117; KIT receptor tyrosine kinase) mediated signalling pathways and (ii) stimulate VEGFR and/or VEGFR mediated signalling pathways. For example, the HE induction medium may comprise SCF and VEGF.

Vascular endothelial growth factor (VEGF) is a protein factor of the PDGF family which binds to VEGFR tyrosine kinase receptors and stimulates vasculogenesis and angiogenesis. Suitable VEGFs include any member of the VEGF family, for example any one of VEGF-A to VEGF-D and PIGF. Preferably, the VEGF is VEGF-A (also known as VEGF, NCBI Gene ID: 7422, nucleic acid sequence NM_001025366.2, amino acid sequence NP_001020537.2). Preferably, the VEGFR and/or VEGFR mediated signalling pathways are VEGFR2 (KDR/Flk-1) and/or VEGFR2 (KDR/Flk-1) mediated signalling pathways. VEGF is readily available from commercial sources (e.g. R&D Systems, USA). Conveniently, the concentration of VEGF in an HE induction medium described herein may be from 1 to 100 ng/ml, for example any of about 5, 7, 10, 12, 15, 17, 20, 25, 30, 35, 40, 45 or 50 ng/ml, preferably about 15 ng/ml.

In some examples of HE induction media, VEGF may be replaced by a VEGF activator or agonist that stimulates VEGFR (or VEGFR2 (KDR/Flk-1)) and/or VEGFR (or VEGFR2 (KDR/Flk-1)) mediated signalling pathways. Suitable VEGF activators are known in the art and include proteins, such as gremlin (Mitola et al., (2010) Blood 116(18) 3677-3680) nucleic acids, such as shRNA (e.g. Turunen et al., Circ Res. 2009 Sep. 11; 105(6):604-9), CRISPR-based plasmids (e.g. VEGF CRISPR activation plasmid; Santa Cruz Biotech, USA), antibodies and small molecules.

Stem cell factor (SCF) is a cytokine that binds to the KIT receptor (KIT proto-oncogene, receptor tyrosine kinase) (CD117; SCFR) and is involved in haematopoiesis. SCF (also called KITLG, NCBI GenelD: 4254) may have the reference nucleic acid sequence NM_000899.5 or NM_03994.5 and the reference amino acid sequence NP_000890.1 or NP_003985.5. SCF is readily available from commercial sources (e.g. R&D Systems, USA). Conveniently, the concentration of SCF in an HE induction medium described herein may be from 1 to 1000 ng/ml, for example any of about 10, 20, 30, 40, 50, 60, 70, 80, 90, 100, 110, 120, 130, 140, 150, 200, 250, 300, 350, 400, 450, 500, 600, 700, 800, 900 ng/ml, preferably about 100 ng/ml.

In preferred embodiments, the HE induction medium is a chemically defined medium. For example, the HE induction medium may consist of a chemically defined nutrient medium supplemented with effective amounts of VEGF, for example 15 ng/ml VEGF; and SCF, for example 100 ng/ml SCF. Preferably, mesoderm cells are cultured in an HE induction medium consisting of a chemically defined nutrient medium and two differentiation factors, wherein the two differentiation factors being SCF and VEGF.

Suitable chemically defined nutrient media are described above and include StemPro™-34 (ThermoFisher Scientific) or a basal medium such as IMDM supplemented with albumin, insulin, selenium transferrin, and lipids as described below.

The mesoderm cells may be cultured in the HE induction medium for 2 to 6 days or 3 to 5 days, preferably about 4 days, to produce a population of HE cells.

Haemogenic endothelial (HE) are partially differentiated endothelial progenitor cells that have hematopoietic potential and are capable of differentiation under appropriate conditions into haematopoietic lineages. HE cells may express CD34 and, in some embodiments, may not express CD73 or CXCR4 (CD184). In some embodiments, the HE cells may have the phenotype CD34+CD73−, or the phenotype CD34+CD73− CXCR4−.

In a third stage, haemogenic endothelial (HE) cells may be differentiated into haematopoietic progenitor cells (HPCs) by culturing the population of HE cells under suitable conditions to promote haematopoietic differentiation. For example, the HE cells may be cultured in a haematopoietic induction medium.

A suitable haematopoietic induction medium may stimulate the following (i) cKIT receptor (CD117; KIT receptor tyrosine kinase) and/or cKIT receptor (CD117; KIT receptor tyrosine kinase) mediated signalling pathways, (ii) VEGFR and/or VEGFR mediated signalling pathways, preferably VEGFR2 and/or VEGFR2 mediated signalling pathways, (iii) MPL (CD110) and/or MPL (CD110) mediated signalling pathways (iv) FLT3 and/or FLT3 mediated signalling pathways (v) IGF1R and/or IGF1R mediated signalling pathways (vi) SMAD1, 5 and 9 and/or SMAD1, 5 and 9 mediated signalling pathways (vii) Hedgehog and/or Hedgehog signalling pathways (viii) EpoR and/or EpoR mediated signalling pathway and (ix) AGTR2 and/or AGTR2 mediated signalling pathways. A suitable haematopoietic induction medium may also inhibit AGTR1 (angiotensin II type 1 receptor (AT₁)) and/or the AGTR1 (angiotensin II type 1 receptor (AT₁)) mediated signaling pathway. A suitable haematopoietic induction medium may also have interleukin (IL) activity and FGF activity.

For example, a haematopoietic induction medium may comprise the differentiation factors: VEGF, SCF, Thrombopoietin (TPO), Flt3 ligand (FIt3L), IL-3, IL-6, IL-7, IL-11, IGF-1, BMP, FGF, Sonic hedgehog (SHH), erythropoietin (EPO), angiotensin II, and an angiotensin II type 1 receptor (AT₁) antagonist. An example of a suitable haematopoietic induction medium is the Stage 3 medium shown in Table 1 below.

Thrombopoietin (TPO) is a glycoprotein hormone that regulates platelet production. TPO (also called THPO, NCBI Gene ID: 7066) may have the reference nucleic acid sequence NM_000460.4 and the reference amino acid sequence NP_000451.1. TPO is readily available from commercial sources (e.g. R&D Systems, USA; Miltenyi Biotec Gmbh, DE). Conveniently, the concentration of TPO in a haematopoietic induction medium described herein may be from 3 to 300 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 100, 110, 120, 130, 140, 150, 160, 170, 180, 190, 200, 210, 220, 230, 240, 250, 260, 270, 280 or 290 ng/ml, preferably about 30 ng/ml.

Flt3 ligand (Fms-related tyrosine kinase 3 ligand or FLT3L) is a cytokine with haematopoietic activity which binds to the FLT3 receptor and stimulates the proliferation and differentiation of progenitor cells. Flt3 ligand (also called FLT3LG, NCBI GenelD: 2323) may have the reference nucleic acid sequence NM_001204502.2 and the reference amino acid sequence NP_001191431.1. Flt3 is readily available from commercial sources (e.g. R&D Systems, USA; Miltenyi Biotec Gmbh, DE). Conveniently, the concentration of Flt3 ligand in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 100, 110, 120, 130, 140, 150, 160, 170, 180, 190, 200, 210, 220, 230 or 240 ng/ml, preferably about 25 ng/ml.

Interleukins (ILs) are cytokines that play major roles in immune development and function. ILs in a haematopoietic induction medium may include IL-3, IL-6, IL-7, and IL-11.

IL-3 (also called IL3 or MCGF, NCBI GenelD: 3562) may have the reference nucleic acid sequence NM_000588.4 and the reference amino acid sequence NP_000579.2. IL-3 is readily available from commercial sources (e.g. R&D Systems, USA; Miltenyi Biotec Gmbh, DE). Conveniently, the concentration of IL-3 in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 100, 110, 120, 130, 140, 150, 160, 170, 180, 190, 200, 210, 220, 230 or 240 ng/ml, preferably about 25 ng/ml.

IL-6 (also called IL6 or HGF, NCBI GenelD: 3569) may have the reference nucleic acid sequence NM_000600.5 and the reference amino acid sequence NP_000591.5. IL-6 is readily available from commercial sources (e.g. R&D Systems, USA; Miltenyi Biotec Gmbh, DE). Conveniently, the concentration of IL-6 in a haematopoietic induction medium described herein may be from 0.1 to 100 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 95 ng/ml, preferably about 10 ng/ml.

IL-7 (also called IL7, NCBI GenelD: 3574) may have the reference nucleic acid sequence NM_000880.4 and the reference amino acid sequence NP_000871.1. IL-7 is readily available from commercial sources (e.g. R&D Systems, USA; Miltenyi Biotec Gmbh, DE). Conveniently, the concentration of IL-7 in a haematopoietic induction medium described herein may be from 0.1 to 100 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 95 ng/ml, preferably about 10 ng/ml.

IL-11 (also called AGIF, NCBI GeneID: 3589) may have the reference nucleic acid sequence NM_000641.4 and the reference amino acid sequence NP_000632.1. IL-11 is readily available from commercial sources (e.g. R&D Systems, USA; Miltenyi Biotec Gmbh, DE). Conveniently, the concentration of IL-11 ligand in a haematopoietic induction medium described herein may be from 0.5 to 100 ng/ml, for example any of about 0.1, 0.25, 0.5, 0.75, 1, 2, 3, 4, 5, 6, 7, 8, 9, 10, 11, 12, 13, 14, 15, 20, 25, 30, 35, 40, 45, 50, 60, 70, 80, 90 or 95 ng/ml, preferably about 5 ng/ml.

Insulin-like growth factor 1 (IGF-1) is a hormone that binds to the tyrosine kinases IGF-1 receptor (IGF1R) and insulin receptor and activates the multiple signalling pathways. IGF-1 (also called IGF or MGF, NCBI GenelD: 3479) may have the reference nucleic acid sequence NM_000618.5 and the reference amino acid sequence NP_000609.1. IGF-1 is readily available from commercial sources (e.g. R&D Systems, USA). Conveniently, the concentration of IGF-1 in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 20, 23, 25, 27, 30, 35, 40, 45, 50, 60, 70, 80, 90 or 100,110,120, 130, 140, 150, 160, 170, 180, 190, 200, 210, 220, 230 or 240 ng/ml, preferably about 25 ng/ml.

Sonic hedgehog (SHH) is a ligand of the hedgehog signalling pathway that regulates vertebrate organogenesis. SHH (also called TPT or HHG1, NCBI GenelD: 6469) may have the reference nucleic acid sequence NM_000193.4 and the reference amino acid sequence NP_000184.1. SHH is readily available from commercial sources (e.g. R&D Systems, USA; Miltenyi Biotec Gmbh, DE). Conveniently, the concentration of SHH in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 20, 23, 25, 27, 30, 35, 40, 45, 50, 60, 70, 80, 90 or 100, 110, 120, 130, 140, 150, 160, 170, 180, 190, 200, 210, 220, 230 or 240 ng/ml, preferably about 25 ng/ml.

Erythropoietin (EPO) is a glycoprotein cytokine that binds to the erythropoietin receptor (EpoR) and stimulates erythropoiesis. EPO (also called DBAL, NCBI GenelD: 2056) may have the reference nucleic acid sequence NM_000799.4 and the reference amino acid sequence NP_000790.2. EPO is readily available from commercial sources (e.g. R&D Systems, USA; PreproTech, USA). Conveniently, the concentration of EPO in haematopoietic induction medium described herein may be from 0.02 to 20 U/ml, for example any of about 0.01, 0.025, 0.05, 0.075, 0.1, 0.5, 0.75, 1.0, 1.5, 2.0, 2.5, 3, 4, 5, 6, 7, 8, 9, 10 13, 15, 17, or 19 U/ml, preferably about 2 U/ml.

Angiotensin II is a heptapeptide hormone that is formed by the action of angiotensin converting enzyme (ACE) on angiotensin I. Angiotensin II stimulates vasoconstriction. Angiotensin I and II are formed by the cleavage of angiotensinogen (also called AGT, NCBI GenelD: 183), which may have the reference nucleic acid sequence NM_000029.4 and the reference amino acid sequence NP_000020.1. Angiotensin II is readily available from commercial sources (e.g. R&D Systems, USA; Tocris, USA). Conveniently, the concentration of Angiotensin II in a haematopoietic induction medium described herein may be from 0.05 to 50 ng/ml, for example any of about 0.01, 0.025, 0.05, 0.075, 0.1, 0.5, 0.75, 1.0, 1.5, 2.0, 2.5, 3, 4, 5, 6, 7, 8, 9, 10, 15, 20, 30, 40 or 50 ng/ml, preferably about 5 ng/ml.

Angiotensin II type 1 receptor (AT₁) antagonists (ARBs) are compounds that selectively block the activation of AT₁ receptor (AGTR1; Gene ID 185). Suitable AT₁ antagonists include losartan (2-Butyl-4-chloro-1-{[2′-(1H-tetrazol-5-yl)-4-biphenylyl]methyl}-1H-imidazol-5-yl)methanol), valsartan ((2S)-3-Methyl-2-(pentanoyl{[2′-(1H-tetrazol-5-yl)biphenyl-4-yl]methyl}amino)butanoic acid), and telmisartan (4′[(1,4′-Dimethyl-2′-propyl[2,6′-bi-1H-benzimidazol]-1′-yl)methyl][1,1′-biphenyl]-2-carboxylic acid. In some preferred embodiments, the AT₁ antagonist is losartan. Suitable AT₁ antagonists may be obtained from commercial suppliers (e.g. Tocris, USA; Cayman Chemical Co. MI USA). Conveniently, the concentration of angiotensin II type 1 receptor (AT₁) antagonist in a haematopoietic induction medium described herein may be from 1 to 1000 μM, for example any of about 10, 20, 30, 40, 50, 60, 70, 80, 90, 100, 110, 120, 130, 140, 150, 160, 170, 180, 190, 200, 250, 300, 350, 400, 450, 500, 600, 700, 800, 900 μM, preferably about 100 μM.

In preferred embodiments, the haematopoietic induction medium is a chemically defined medium. For example, the haematopoietic induction medium may consist of a chemically defined nutrient medium supplemented with effective amounts of VEGF, for example 15 ng/ml; SCF, for example 100 ng/ml; thrombopoietin (TPO), for example 30 ng/ml; Flt3 ligand (FLT3L), for example 25 ng./ml; IL-3, for example 25 ng/ml; IL-6, for example 10 ng/ml; IL-7, for example 10 ng/ml; IL-11, for example 5 ng/ml; IGF-1, for example 25 ng/ml; BMP, for example BMP4 at 10 ng/ml; FGF, for example bFGF at 5 ng/ml; Sonic hedgehog (SHH), for example 25 ng/ml; erythropoietin (EPO), for example 2 u/ml; angiotensin II, for example 10 μg/ml, and an angiotensin II type 1 receptor (AT₁) antagonist, for example losartan, at 100 μM.

Suitable chemically defined nutrient media are described above and include StemPro™-34 PLUS (ThermoFisher Scientific) or a basal medium such as IMDM supplemented with albumin, insulin, selenium transferrin, and lipids as described below.

The HE cells may be cultured in the haematopoietic induction medium for 8-21 days, for example any of about 9, 10, 11, 12, 13, 14, 15, 16, 17, 18, 19 or 20 days, preferably about 16 days, to produce the population of HPCs.

Following the generation of HPCs from HE cells, a population of HPCs expressing one or more cell surface markers, such as CD34, may be purified, for example by magnetic activated cell sorting (MACS), before being subjected to further differentiation. For example, a population of CD34+ HPCs may be purified. The CD34+ HPCs may be purified after 8 days, for example 8, 9 or 10 days, culture in the HE induction medium. The CD34+ HPCs may be purified after 16 days of differentiation, for example on day 16, 17 or 18 of the differentiation method.

Following production by a method described above, the population of progenitor T cells may be expanded. For example, a population of progenitor T cells may be cultured under suitable conditions to promote an increase in the number of progenitor T cells in the population. Suitable conditions may include the presence of a pyrimidoindole compound.

For example, the population of progenitor T cells may be cultured in a lymphoid expansion medium supplemented with the pyrimidoindole compound, as described above.

Following production by a method described above and optional expansion, the progenitor T cells may be matured or further differentiated into immune cells, such as TCR αβ+ T cells, TCR γδ+ T cells, NK cells and NKT cells.

In some preferred embodiments, the progenitor T cells may be matured into TCR αβ+ T cells. For example, a population of progenitor T cells may be cultured under suitable conditions to promote T cell maturation. Suitable conditions may include the absence of a pyrimidoindole compound.

In some embodiments, the progenitor T cells may be cultured in a T cell maturation medium. A T cell maturation medium is a cell culture medium that promotes the maturation of progenitor T cells into mature T cells. A suitable T cell maturation medium may (i) stimulate cKIT receptor (CD117; KIT receptor tyrosine kinase) and/or cKIT receptor (CD117; KIT receptor tyrosine kinase) mediated signalling pathways (ii) FLT3 and/or FLT3 mediated signalling pathways and (iii) have interleukin (IL) activity. For example, a T cell maturation medium may comprise the differentiation factors SCF, FLT3L, and IL7.

Preferably, the T cell maturation medium does not contain a pyrimidoindole compound. For example, following differentiation and optional expansion of the population of progenitor T cells in the presence of a pyrimidoindole compound. as described herein, the population may be matured, activated and/or expanded in the absence of the pyrimidoindole compound.

In preferred embodiments, the T cell maturation medium is a chemically defined medium. For example, the T cell maturation medium may consist of a chemically defined nutrient medium supplemented with effective amounts of the above differentiation factors. Suitable T cell maturation media are well-known in the art and include Stemspan™ SFEM II (Cat #9605; StemCell Technologies Inc, CA) with Stemspan™ T cell maturation supplement (Cat #9930; StemCell Technologies Inc, CA) and other media suitable for expansion of PBMCs and CD3+ cells, such as ExCellerate Human T cell expansion medium (R& D Systems, USA). Other suitable T cell maturation media may include a basal medium such as IMDM, supplemented with ITS, albumin and lipids, as described elsewhere herein and further supplemented with effective amounts of the above differentiation factors.

The progenitor T cells may be cultured on a surface. For example, the progenitor T cells may be cultured on a surface of a culture vessel, bead or other biomaterial or polymer. Preferably, the surface may be coated with a factor that stimulates Notch signalling, for example a Notch ligand, such as Delta-like 1 (DLL1) or Delta-like 4 (DLL4). Suitable Notch ligands are well-known in the art and available from commercial suppliers. The surface may also be coated with an extracellular matrix protein, such as fibronectin, vitronectin, laminin or collagen and/or one or more cell surface adhesion proteins, such as VCAM1. Suitable coatings are well-known in the art and described elsewhere herein.

The progenitor T cells may be cultured in the T cell maturation medium on the substrate for a time sufficient for the progenitor T cells to mature into TCR αβ+ T cells. For example, the progenitor T cells may be cultured for 1-4 weeks, preferably 2 or 3 weeks.

TCR αβ+ T cells produced as described herein may be mature CD3+ T cells. For example, the cells may have a αβTCR+CD3+CD45+CD28+ phenotype.

In some embodiments, the TCR αβ+ T cells produced by maturation of progenitor T cells (stage 5) may be or may predominantly be double positive CD4+CD8+ T cells.

In a sixth stage, the population of TCR αβ+ T cells may be activated and/or expanded to produce or increase the proportion of single positive CD4+ T cells, or more preferably single positive CD8+ T cells. TCR αβ+ T cells produced by the methods described herein may be double positive CD4+CD8+ T cells or single positive CD4+ or CD8+ T cells.

Suitable methods for activating and expanding T cells are well-known in the art. For example, T cells may be exposed to a T cell receptor (TCR) agonist under appropriate culture conditions. Suitable TCR agonists include ligands, such as peptides displayed on a class I or II MHC molecules (MHC-peptide complexes) on the surface of a bead or an antigen presenting cell, such as a dendritic cell, and soluble factors, such as anti-TCR antibodies, for example anti-CD28 antibodies, and multimeric MHC-peptide complexes, such as MHC-peptide tetramers, pentamers or dextramers.

Activation refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

An anti-TCR antibody may specifically bind to a component of the TCR, such as εCD3, αCD3 or αCD28. Anti-TCR antibodies suitable for TCR stimulation are well-known in the art (e.g. OKT3) and available from commercial suppliers (e.g. eBioscience CO USA). In some embodiments, T cells may be activated by exposure to anti-aCD3 antibodies and IL2, IL-7 or IL15. More preferably, T cells are activated by exposure to anti-aCD3 antibodies and anti-aCD28 antibodies. The activation may occur in the presence or absence of CD14⁺ monocytes. The T cells may be activated with anti-CD3 and anti-CD28 antibody coated beads. For example, PBMCs or T cell subsets including CD4⁺ and/or CD8⁺ cells may be activated, without feeder cells (antigen presenting cells) or antigen, using antibody coated beads, for example magnetic beads coated with anti-CD3 and anti-CD28 antibodies, such as Dynabeads® Human T-Activator CD3/CD28 (ThermoFisher Scientific). In other embodiments, soluble tetrameric antibody complexes that bind CD3, CD28 and CD2 cell surface ligands, such as ImmunoCult™ Human CD3/CD28/CD2 T Cell Activator or Human CD3/CD28 T Cell Activator, may be used to activate the T cells. In other embodiments, T cells may be activated with an MHC-peptide complex, preferably a multimeric MHC-peptide complex, optionally in combination with an anti-CD28 antibody.

In some embodiments, TCR αβ+ T cells, for example double positive CD4+CD8+ T cells, may be cultured in a T cell maturation medium as described herein supplemented with IL-15. The medium may be further supplemented with a T cell receptor (TCR) agonist, for example one or more anti-TCR antibodies, such as anti-aCD3 antibodies, and anti-aCD28 antibodies, as described above.

The TCR αβ+ T cells may be cultured using any convenient technique to produce the expanded population. Suitable culture systems include stirred tank fermenters, airlift fermenters, roller bottles, culture bags or dishes, and other bioreactors, in particular hollow fibre bioreactors. The use of such systems is well-known in the art

TCR αβ+ T cells produced as described herein may express an αβ TCR that binds a target antigen. For example, the αβ TCR may bind specifically to cancer cells that express a tumor antigen. The T cells may be useful for example in immunotherapy, as described below.

In some embodiments, the αβTCR expressed by the T cells may be naturally expressed (i.e. an endogenous TCR). For example, the T cells may be produced as described herein from iPSCs that are derived from Tumour Infiltrating Lymphocytes (TILs). TILs, for example tumour resident CD3+CD8+ cells, may be obtained from an individual with a cancer condition using standard techniques. Alternatively, the T cells may be produced as described herein from iPSCs that are derived from T cells that bind to a peptide fragment of the target antigen displayed on a class I or II MHC molecule on the surface of an antigen presenting cell, such as a dendritic cell; or a population of T cells produced as described herein may be screened for binding to a peptide fragment of the target antigen displayed on a class I or II MHC molecule, and T cells that bind to the displayed peptide fragment identified.

In other embodiments, the αβ TCR is not naturally expressed by the cells (i.e. the TCR is exogenous or heterologous). Suitable heterologous αβTCR may bind specifically to class I or II MHC molecules displaying peptide fragments of a target antigen. For example, the T cells may be modified to express a heterologous αβTCR that binds specifically to class I or II MHC molecules displaying peptide fragments of a tumour antigen expressed by the cancer cells in a cancer patient. In some embodiments the TCR may recognise a target antigen or a peptide fragment of a target antigen on the cancer cell independently of MHC presentation. Tumour antigens expressed by cancer cells in the cancer patient may identified using standard techniques. Preferred tumour antigens include NY-ESO1, PRAME, alpha-fetoprotein (AFP), MAGE A4, MAGE A1, MAGE A10 and MAGE B2, most preferably NY-ESO-1, MAGE-A4 and MAGE-A10.

A heterologous TCR may be a synthetic or artificial TCR i.e. a TCR that does not exist in nature. For example, a heterologous TCR may be engineered to increase its affinity or avidity for a tumour antigen (i.e. an affinity enhanced TCR). The affinity enhanced TCR may comprise one or more mutations relative to a naturally occurring TCR, for example, one or more mutations in the hypervariable complementarity determining regions (CDRs) of the variable regions of the TCR α and β chains. These mutations increase the affinity of the TCR for MHCs that display a peptide fragment of a tumour antigen expressed by cancer cells. Suitable methods of generated affinity enhanced TCRs include screening libraries of TCR mutants using phage or yeast display and are well known in the art (see for example Robbins et al., J Immunol (2008) 180(9):6116; San Miguel et al., (2015) Cancer Cell 28 (3) 281-283; Schmitt et al., (2013) Blood 122 348-256; Jiang et al., (2015) Cancer Discovery 5 901). Preferred affinity enhanced TCRs may bind to cancer cells expressing one or more of the tumour antigens NY-ESO1, PRAME, alpha-fetoprotein (AFP), MAGE A4, MAGE A1, MAGE A10 and MAGE B2.

Expression of a heterologous as TCR may alter the immunogenic specificity of the T cells produced as described herein so that they recognise or display improved recognition for one or more target antigens, e.g. tumour antigens that are present on the surface of the cancer cells of an individual with cancer. In some embodiments, the T cells produced as described herein may display reduced binding or no binding to cancer cells in the absence of the heterologous αβTCR. For example, expression of the heterologous αβ TCR may increase the affinity and/or specificity of the cancer cell binding of a T cell relative to T cells that do not express the as TCR.

The term “heterologous” refers to a polypeptide or nucleic acid that is foreign to a particular biological system, such as a host cell, and is not naturally present in that system. A heterologous polypeptide or nucleic acid may be introduced to a biological system by artificial means, for example using recombinant techniques. For example, heterologous nucleic acid encoding a polypeptide may be inserted into a suitable expression construct which is in turn used to transform a host cell to produce the polypeptide. A heterologous polypeptide or nucleic acid may be synthetic or artificial or may exist in a different biological system, such as a different species or cell type. An endogenous polypeptide or nucleic acid is native to a particular biological system, such as a host cell, and is naturally present in that system. A recombinant polypeptide is expressed from heterologous nucleic acid that has been introduced into a cell by artificial means, for example using recombinant techniques. A recombinant polypeptide may be identical to a polypeptide that is naturally present in the cell or may be different from the polypeptides that are naturally present in that cell.

T cells may be modified to express the heterologous as TCR by the introduction of heterologous encoding nucleic acid into cells at any stage in the method described herein. For example, heterologous encoding nucleic acid may be introduced into iPSCs, HPCs or progenitor T cells. In some preferred embodiments, cells may be transduced with heterologous nucleic acid encoding an αβTCR after culture in lymphoid expansion medium, for example after 2 weeks culture in lymphoid expansion medium (stage 4) as described herein. Heterologous nucleic acid encoding a TCR may encode all the sub-units of the receptor. For example, nucleic acid encoding a TCR may comprise a nucleotide sequence encoding a TCR α chain and a nucleotide sequence encoding a TCR β chain.

Nucleic acid may be introduced into the cells by any convenient technique. When introducing or incorporating a heterologous nucleic acid into an iPSC, HPC or progenitor T cell, certain considerations must be taken into account, well-known to those skilled in the art. The nucleic acid to be inserted should be assembled within a construct or vector which contains effective regulatory elements which will drive transcription in the T cell. Many known techniques and protocols for manipulation and transformation of nucleic acid, for example in preparation of nucleic acid constructs, introduction of DNA into cells and gene expression are described in detail in Protocols in Molecular Biology, Second Edition, Ausubel et al. eds. John Wiley & Sons, 1992. In some embodiments, nucleic acid may be introduced into the cells by gene editing. For example, a DNA double strand break (DSB) at a target site may be induced by a CRISPR/Cas9 system and the repair of the DSB may introduce the heterologous nucleic acid into the cell genome at the target site or the nucleic acid may be introducing using an rAAV vector (AAV mediated gene editing; Hirsch et al., 2014 Methods Mol Biol 1114 291-307).

Suitable techniques for introducing the expression vector into the iPSCs, HPCs or progenitor T cells are well known in the art and include calcium phosphate transfection, DEAE-Dextran, electroporation, liposome-mediated transfection, gene editing and transduction using retrovirus or other virus, e.g. vaccinia or lentivirus. Preferably, nucleic acid encoding the heterologous as TCR may be contained in a viral vector, most preferably a gamma retroviral vector or a lentiviral vector, such as a VSVg-pseudotyped lentiviral vector. A method described herein may comprise transducing a population of cells, for example iPSCs, HPCs or progenitor T cells, with a viral vector to produce a transduced population of genetically modified cells. The cells may be transduced by contact with a viral particle comprising the nucleic acid. Viral particles for transduction may be produced according to known methods. For example, HEK293T cells may be transfected with plasmids encoding viral packaging and envelope elements as well as a lentiviral vector comprising the coding nucleic acid. A VSVg-pseudotyped viral vector may be produced in combination with the viral envelope glycoprotein G of the Vesicular stomatitis virus (VSVg) to produce a pseudotyped virus particle. For example, solid-phase transduction may be performed without selection by culture on retronectin-coated, retroviral vector-preloaded tissue culture plates.

Following production, the population of TCR αβ+ T cells, for example DP CD4+CD8+ cells, SP CD4+ cells or SP CD8+ cells, may be isolated and/or purified. Any convenient technique may be used, including fluorescence-activated cell sorting (FACS) or magnetic-activated cell sorting using antibody coated magnetic particles (MACS).

The population of TCR αβ+ T cells, for example DP CD4+CD8+ cells, SP CD4+ cells or SP CD8+ cells, may be expanded and/or concentrated. Optionally, the population of TCR αβ+ T cells produced as described herein may be stored, for example by cryopreservation, before use.

A population of TCR αβ+ T cells may be admixed with other reagents, such as buffers, carriers, diluents, preservatives and/or pharmaceutically acceptable excipients. Suitable reagents are described in more detail below. A method described herein may comprise admixing the population of TCR αβ+ T cells with a pharmaceutically acceptable excipient.

Pharmaceutical compositions suitable for administration (e.g. by infusion), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable vehicles can be found in standard pharmaceutical texts, for example, Remington's Pharmaceutical Sciences, 18th edition, Mack Publishing Company, Easton, Pa., 1990.

In some preferred embodiments, the TCR αβ+ T cells, which may be DP CD4+CD8+ T cells, SP CD4+ T cells or preferably SP CD8+ T cells, may be formulated into a pharmaceutical composition suitable for intravenous infusion into an individual.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

An aspect of the invention provides a population of TCR αβ+ T cells, which may be for example DP CD4+CD8+ T cells, SP CD4+ T cells or SP CD8+ T cells, produced by a method described above.

The population of TCR αβ+ T cells may be for use as a medicament. For example, a population of mature TCR αβ+ T cells as described herein may be used in cancer immunotherapy therapy, for example adoptive T cell therapy.

Adoptive cellular therapy or adoptive immunotherapy refers to the adoptive transfer of human T lymphocytes that express TCRs that are specific for antigens or peptides thereof expressed on target cells and/or TCRs that are specific for peptide MHC complexes expressed on target cells.

This can be used to treat a range of diseases depending upon the target chosen, e.g., tumour specific antigens to treat cancer. Adoptive cellular therapy involves removing a portion of a donor's or the patient's cells, for example, white blood cells. The cells are then used to generate iPSCs in vitro and these iPSCs are used to efficiently generate T cells that are specific for antigens or peptides thereof expressed on target cells and/or specific for peptide MHC complexes on target cells as described herein. The T cells may be expanded, washed, concentrated, and/or then frozen to allow time for testing, shipping and storage until a patient is ready to receive the infusion of cells.

Other aspects of the invention provide the use of a population of TCR αβ+ T cells as described herein for the manufacture of a medicament for the treatment of cancer, a population of TCR αβ+ T cells as described herein for the treatment of cancer, and a method of treatment of cancer comprising administering a population of TCR αβ+ T cells as described herein to an individual in need thereof.

The population of TCR αβ+ T cells may be autologous i.e. the TCR αβ+ T cells were originally obtained from the same individual to whom they are subsequently administered (i.e. the donor and recipient individual are the same).

The population of TCR αβ+ T cells may be allogeneic i.e. the TCR αβ+ T cells may be originally derived from a different individual to the individual to whom they are subsequently administered (i.e. the donor and recipient individual are different) i.e. the individual from whom the cells used to generate the iPSCs were obtained different to the individual to whom the TCR αβ+ T cells are administered. Allogeneic refers to a graft derived from a different animal of the same species.

The donor and recipient individuals may be HLA matched to avoid GVHD and other undesirable immune effects, such as rejection. Alternatively, the donor and recipient individuals may not be HLA matched, or HLA genes in the cells from the donor individual may be modified, for example by gene editing, to remove any HLA mismatch with the recipient.

A suitable population of TCR αβ+ T cells for administration to a recipient individual may be produced by a method comprising providing an initial population of cells, preferably T cells, obtained from a donor individual, reprogramming the cells into iPSCs and differentiating the iPSCs into T cells that express an αβTCR which binds specifically to cancer cells and/or an antigen or peptide thereof presented by cancer cells optionally in complex with MHC, in the recipient individual.

Following administration of the TCR αβ+ T cells, the recipient individual may exhibit a T cell mediated immune response against cancer cells in the recipient individual. This may have a beneficial effect on the cancer condition in the individual.

As used herein, the terms “cancer,” “neoplasm,” and “tumour” are used interchangeably and, in either the singular or plural form, refer to cells that have undergone a malignant transformation that makes them pathological to the host organism.

Primary cancer cells can be readily distinguished from non-cancerous cells by well-established techniques, particularly histological examination. A cancer cell may include not only a primary cancer cell, but any cell derived from a cancer cell ancestor. This includes metastasized cancer cells, and in vitro cultures and cell lines derived from cancer cells. When referring to a type of cancer that normally manifests as a solid tumour, a “clinically detectable” tumour is one that is detectable on the basis of tumour mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient.

Cancer conditions may be characterised by the abnormal proliferation of malignant cancer cells and may include leukaemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, oesophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

Cancer cells within an individual may be immunologically distinct from normal somatic cells in the individual (i.e. the cancerous tumour may be immunogenic). For example, the cancer cells may be capable of eliciting a systemic immune response in the individual against one or more antigens expressed by the cancer cells. The tumour antigens that elicit the immune response may be specific to cancer cells or may be shared by one or more normal cells in the individual.

The cancer cells of an individual suitable for treatment as described herein may express the antigen and/or may be of correct HLA type to bind the as TCR expressed by the T cells.

An individual suitable for treatment as described above may be a mammal. In preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

In some embodiments, the individual may have minimal residual disease (MRD) after an initial cancer treatment.

An individual with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a cancer in an individual may include identification of a particular cell type (e.g. a cancer cell) in a sample of a body fluid or tissue obtained from the individual.

An anti-tumour effect is a biological effect which can be manifested by a reduction in the rate of tumour growth, decrease in tumour volume, a decrease in the number of tumour cells, a decrease in the number of metastases, an increase in life expectancy, or amelioration of various physiological symptoms associated with the cancerous condition. An “anti-tumour effect” can also be manifested by the ability of the peptides, polynucleotides, cells, particularly T cells produced according to the methods of the present invention, and antibodies described herein in prevention of the occurrence of tumour in the first place Treatment may be any treatment and/or therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment may also be prophylactic (i.e. prophylaxis). For example, an individual susceptible to or at risk of the occurrence or re-occurrence of cancer may be treated as described herein. Such treatment may prevent or delay the occurrence or re-occurrence of cancer in the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumour volume or morphology (for example, as determined using computed tomographic (CT), sonography, or other imaging method), a delayed tumour growth, a destruction of tumour vasculature, improved performance in delayed hypersensitivity skin test, an increase in the activity of T cells, and a decrease in levels of tumour-specific antigens. Administration of T cells modified as described herein may improve the capacity of the individual to resist cancer growth, in particular growth of a cancer already present the subject and/or decrease the propensity for cancer growth in the individual.

The TCR αβ+ T cells or the pharmaceutical composition comprising the TCR αβ+ T cells may be administered to a subject by any convenient route of administration, whether systemically/peripherally or at the site of desired action, including but not limited to; parenteral, for example, by infusion. Infusion involves the administration of the T cells in a suitable composition through a needle or catheter. Typically, T cells are infused intravenously or subcutaneously, although the T cells may be infused via other non-oral routes, such as intramuscular injections and epidural routes. Suitable infusion techniques are known in the art and commonly used in therapy (see, e.g., Rosenberg et al., New Eng. J. of Med., 319:1676, 1988).

Typically, the number of cells administered is from about 105 to about 10¹⁰ per Kg body weight, for example any of about 1, 2, 3, 4, 5, 6, 7, 8, or 9, ×10⁵, ×106, ×10⁷, ×10⁸, ×109, or ×10¹⁰ cells per individual, typically 2×10⁸ to 2×10¹⁰ cells per individual, typically over the course of 30 minutes, with treatment repeated as necessary, for example at intervals of days to weeks. It will be appreciated that appropriate dosages of the TCR αβ+ T cells, and compositions comprising the TCR αβ+ T cells, can vary from patient to patient.

Determining the optimal dosage will generally involve the balancing of the level of therapeutic benefit against any risk or deleterious side effects of the treatments of the present invention. The selected dosage level will depend on a variety of factors including, but not limited to, the activity of the particular cells, cytokine release syndrome (CRS), the route of administration, the time of administration, the rate of loss or inactivation of the cells, the duration of the treatment, other drugs, compounds, and/or materials used in combination, and the age, sex, weight, condition, general health, and prior medical history of the patient. The amount of cells and the route of administration will ultimately be at the discretion of the physician, although generally the dosage will be to achieve local concentrations at the site of action which achieve the desired effect without causing substantial harmful or deleterious side-effects.

While the TCR αβ+ T cells may be administered alone, in some circumstances the TCR αβ+ T cells may be administered cells in combination with the target antigen, APCs displaying the target antigen, CD3/CD28 beads, IL-2, IL7 and/or IL15 to promote expansion in vivo of the population of TCR αβ+ T cells. Administration in combination may be by separate, simultaneous or sequential administration of the combined components.

The population of TCR αβ+ T cells may be administered in combination with one or more other therapies, such as cytokines e.g. IL-2, CD4+CD8+ chemotherapy, radiation and immuno-oncology agents, including checkpoint inhibitors, such as anti-B7-H3, anti-B7-H4, anti-TIM3, anti-KIR, anti-LAG3, anti-PD-1, anti-PD-L1, and anti-CTLA4 antibodies. Administration in combination may be by separate, simultaneous or sequential administration of the combined components.

The one or more other therapies may be administered by any convenient means, preferably at a site which is separate from the site of administration of the TCR αβ+ T cells.

Administration of TCR αβ+ T cells can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. Preferably, the TCR αβ+ T cells are administered in a single transfusion, for example of any of 500 million, 1 billion, 2, 3, 4, 5, 6, 7, 8, 9, 10, 11, 12, 13, 14, 15 billion T cells for example at least 1×10⁹ T cells.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

### EXPERIMENTAL

### Materials and Methods

### UM729 Preparation

A stock solution of 5 mM of UM729 was made by reconstitution in DMSO. This stock solution was stored at −20° C. Working stock solutions were made on the day of the experiment at 1 mM in DMSO and used to supplement the media at the relevant concentrations (0.25 μM-5 μM)

iProT cell Differentiation from Pluripotent Stem Cells

iPSC cells (HCP1324_c1A12) were differentiated to day 16, using our in-house Proprietary media. HPC (CD34+ cells) were isolated using MACS and a cryopreserved bank of these cells was created for further differentiation into iT-Cells.

HPC were then thawed and seeded at 5000 cells per cm² in 1 ml of lymphoid progenitor (stage 4; LP) media on the standard SCT's coating (2.5 μL/cm²). UM729 was added to the culture media in a range of concentrations from 0.25 μM-5 μM. On day 3, 1 mL of LP media with the required concentration of UM729 was added and further demi-media changes were carried out every 3 days, with the required media concentration. Every 14 days cells were removed from culture and counted using NC-250. The cells were then replated on to fresh SCT matrix and UM729 was added to the media at the relevant concentration. Cells were removed from the cultures after 21, 35 and 42 days and phenotyped using the panel in Table 1. At these timepoints cells were either continued on in lymphoid progenitor media with the relevant UM729 concentrations or transferred into T cell maturation conditions.

### Maturation of T Cells

On day 21, 35 and 42 of culture iPro T Cells were seeded onto fresh SCT matrix (2.5 μl/cm²) at a density of 250,000 cells per cm². Cells were cultured in T-Cell maturation media either with or without UM729 (0.25-5 μM), with demi-media changes every 3 days for 2 weeks. After 2 weeks of maturation the cells were counted using the NC-250 and phenotyped by flow cytometry using the panel in Table 1.

### Cell Phenotyping

On the day of the assay, the cells were counted, washed twice in FACS Buffer and phenotyped by flow cytometry for T-cell markers. The list of antibodies used can be found in Table 1.

### Results

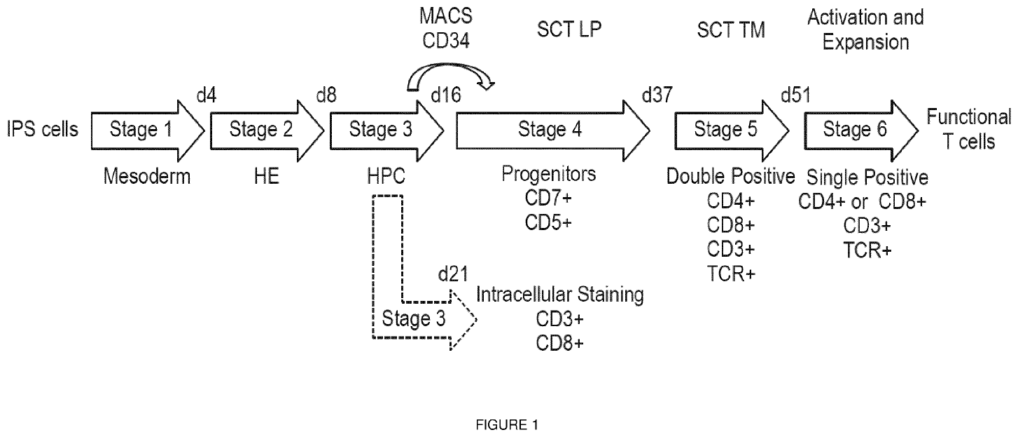

The effect of UM729 and UM171 was assessed in both stage 4 and stage 5 of the six-stage differentiation process (FIG. 1). In stage 4 of the differentiation process, HPCs isolated from stage 3 are proliferated and further differentiated into Pro T cells, which are CD5+CD7+. In stage 5 of the process, these Pro T cells are matured into CD4+CD8+αβ T-Cells.

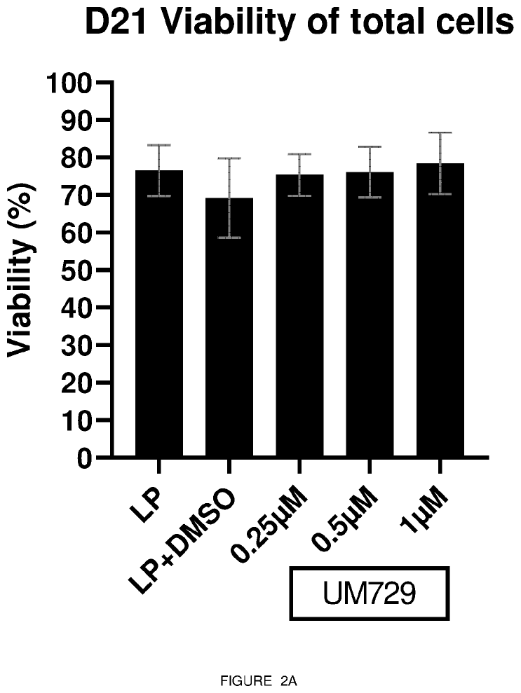

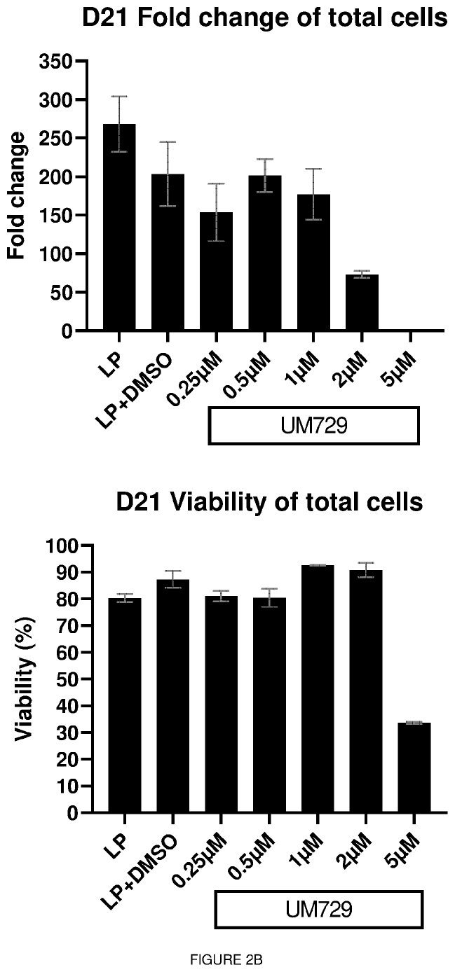

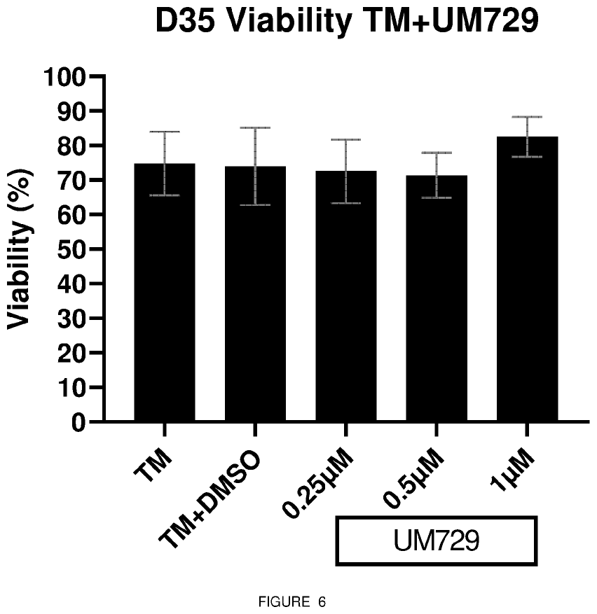

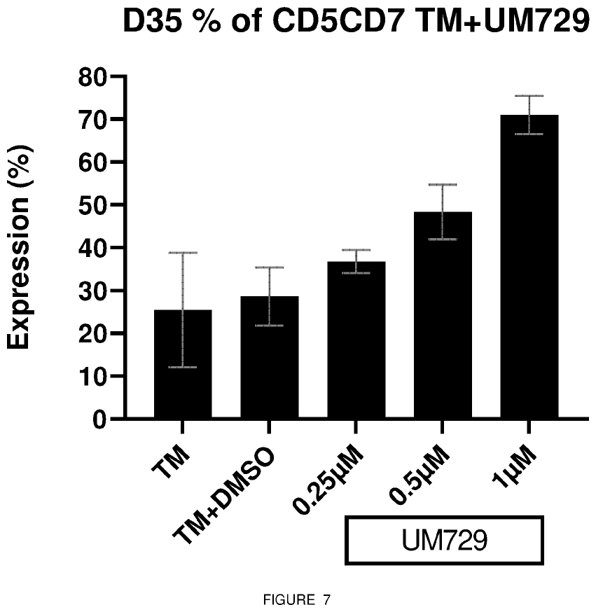

The addition of UM729 in either stage 4, lymphoid proliferation media (LP), or stage 5, T-cell maturation Media™, up to a concentration of 1 μM was shown not to affect the viability of the iPro T Cells after 21 days and 35 days in culture (FIG. 2A and FIG. 6). However, cytotoxic effects were observed at concentrations of 5 μM and above, the viability of total cells was comparably low and proliferation of total cells ceased when treating cells at a concentration of 5 μM for 21 days. (FIG. 2B).

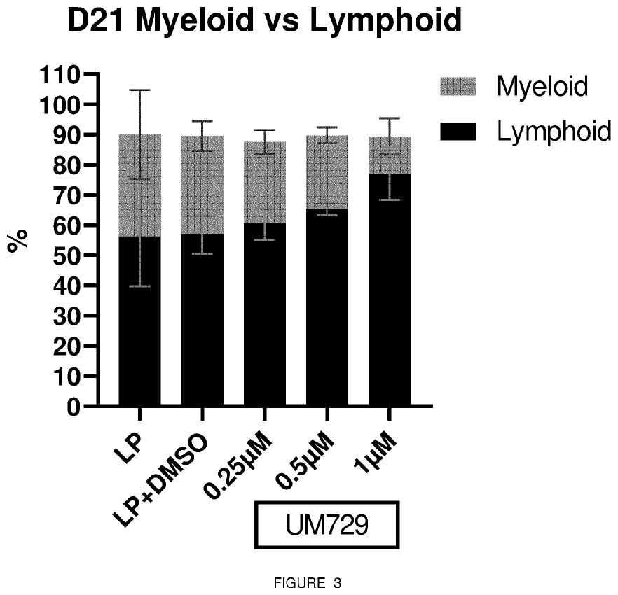

HPCs cultured into lymphoid progenitors also have the potential to differentiate into myeloid cells (FIG. 3) The addition of UM729 was determined by flow cytometry to reduce this cellular contamination in a dose dependent manner (FIG. 3).

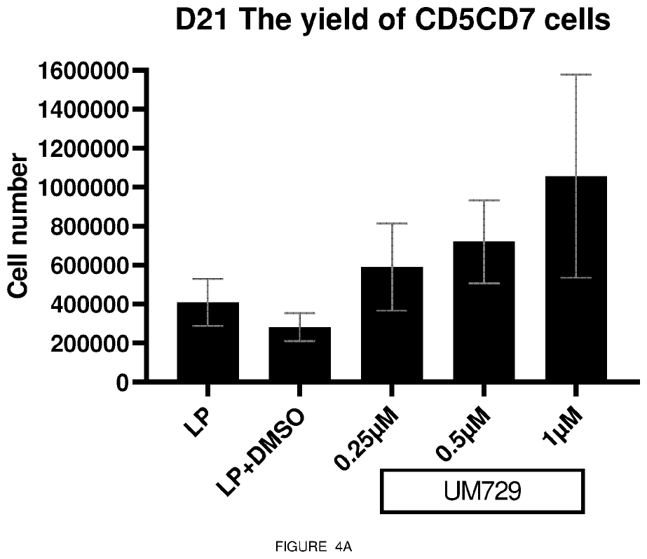

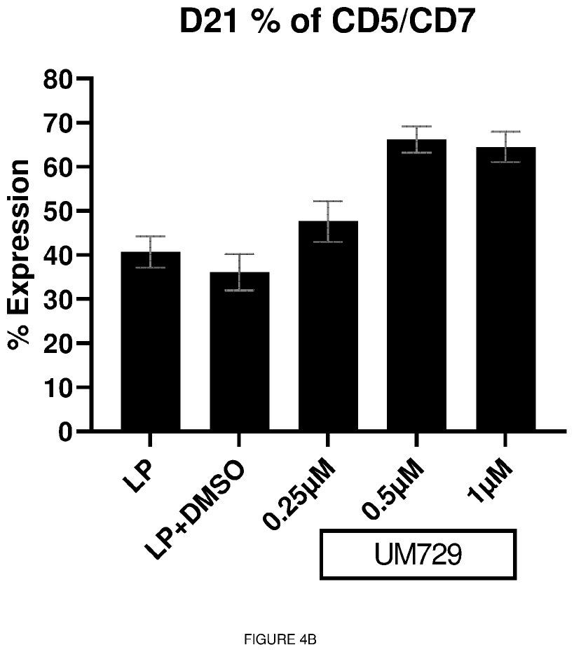

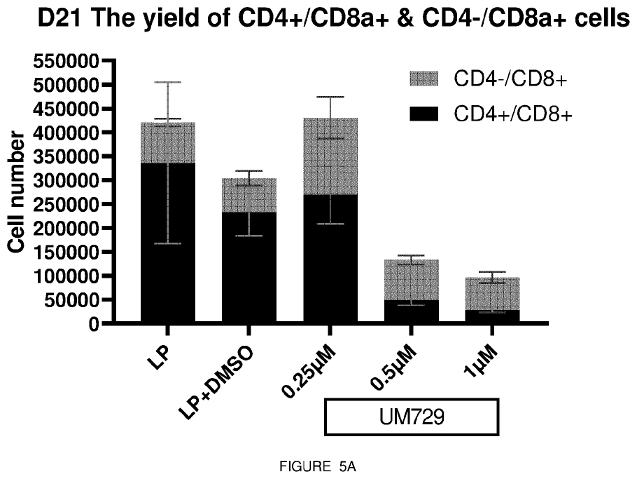

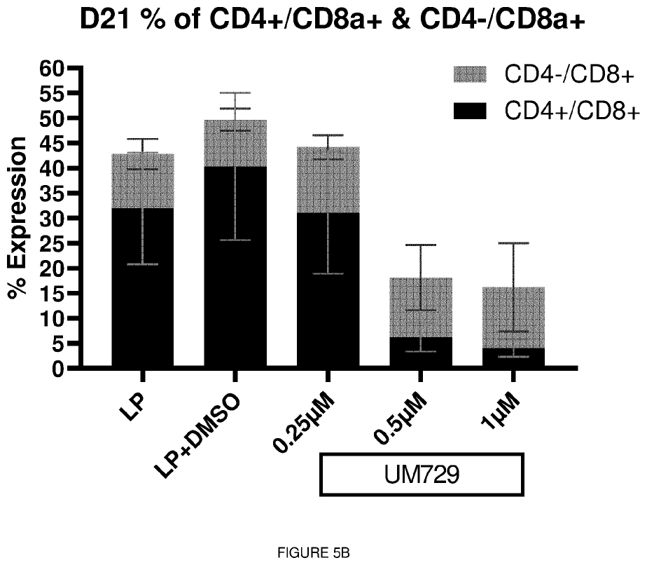

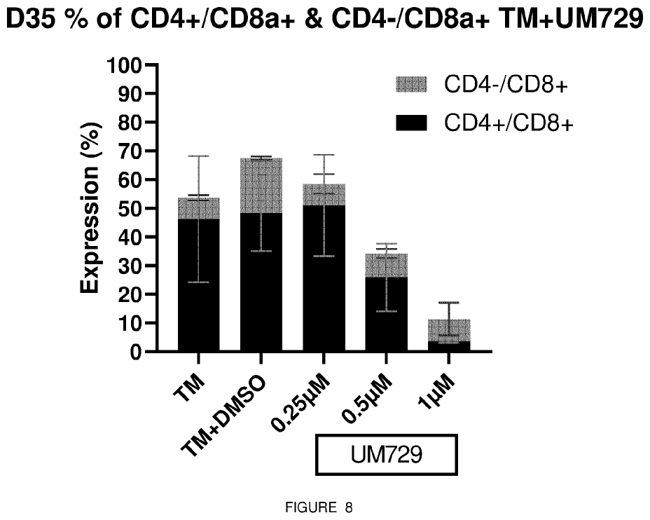

The addition of UM729 to stage 4 media was found to increase the yield of CD7+CD5+ iPro T Cells (FIG. 4A) and also to enrich the percentage of CD7+CD5+ iPro T Cells (FIG. 4B). However, the addition of UM729 to stage 4 media led to a developmental block which inhibited further differentiation through immature single positive CD4+ T Cells to CD4+CD8+ double positive T-Cells (FIG. 5A and FIG. 5B). This developmental block allowed the synchronisation and enrichment of the differentiation cultures at the iPro T cell stage. The developmental block continued if UM729 was also used in the stage 5 cultures, with the percentage of CD7+CD5+ iPro T Cells within the cultures increasing and the development of CD4+CD8+ being inhibited in a dose dependent manner (FIG. 7 and FIG. 8).

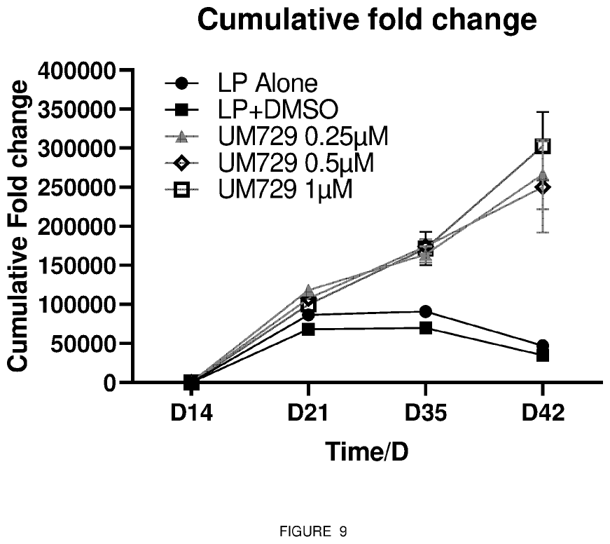

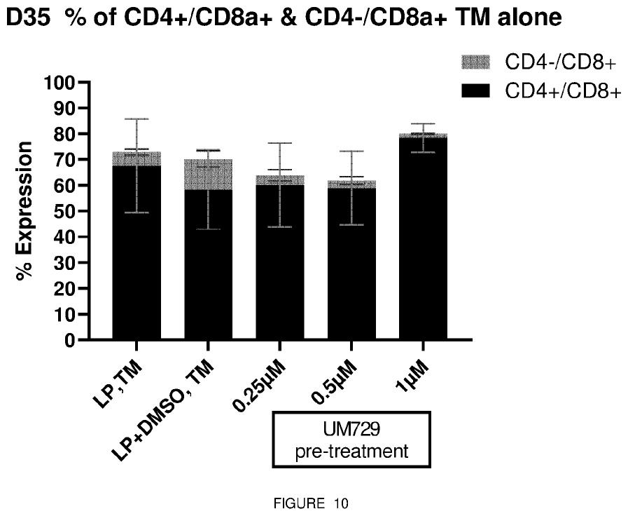

The developmental block caused by the presence of UM729 was used to continually expand the cells in stage 4, with expansion of iPro T cells being supported over 42 days, compared to cultures in the absence of UM729 which fail to maintain their proliferative capacity past day 21 (FIG. 9). Importantly the developmental block was found to be removed by the removal of UM729 in T-Cell maturation media, allowing for the differentiation of CD4+CD8+αβ T Cells (FIG. 10).

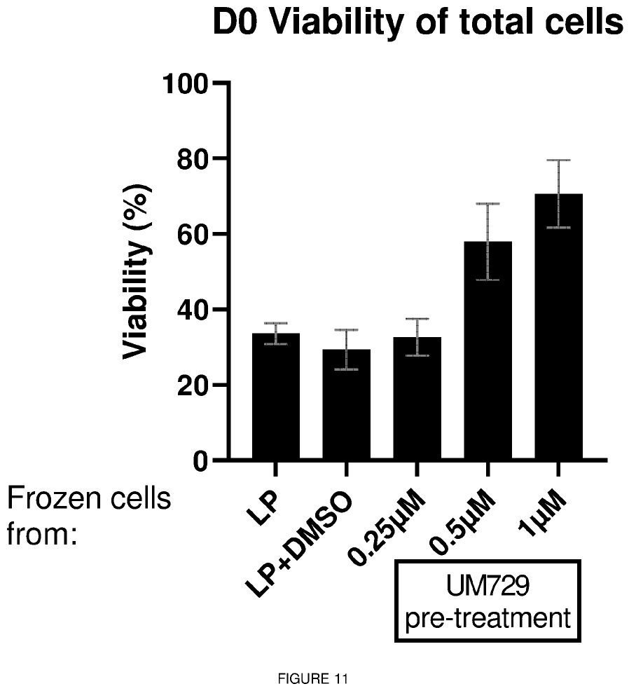

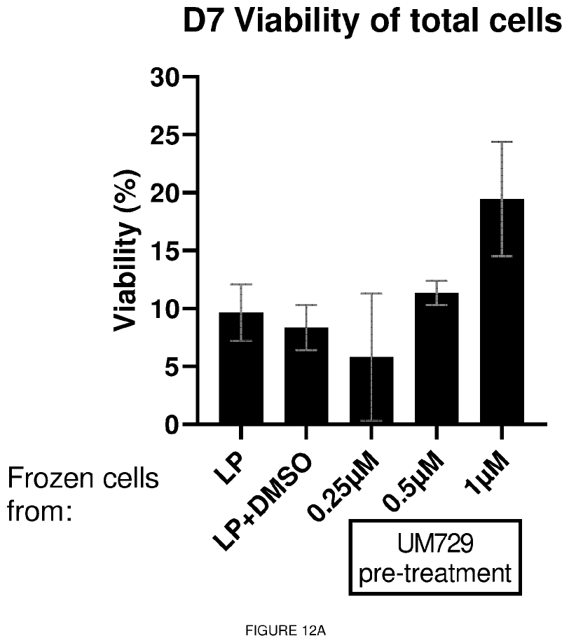

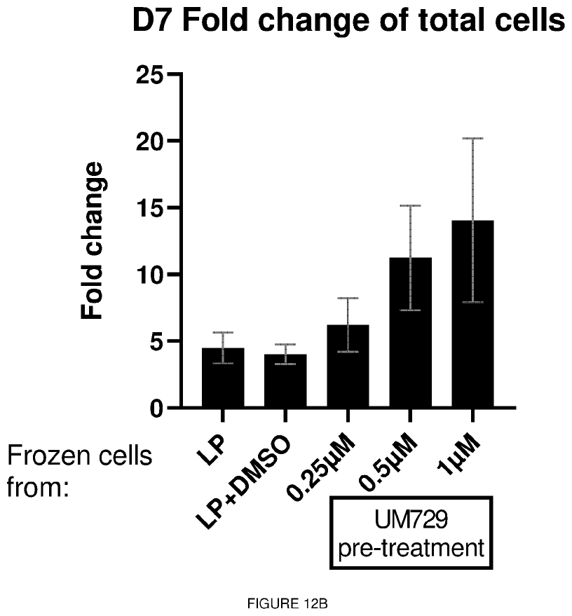

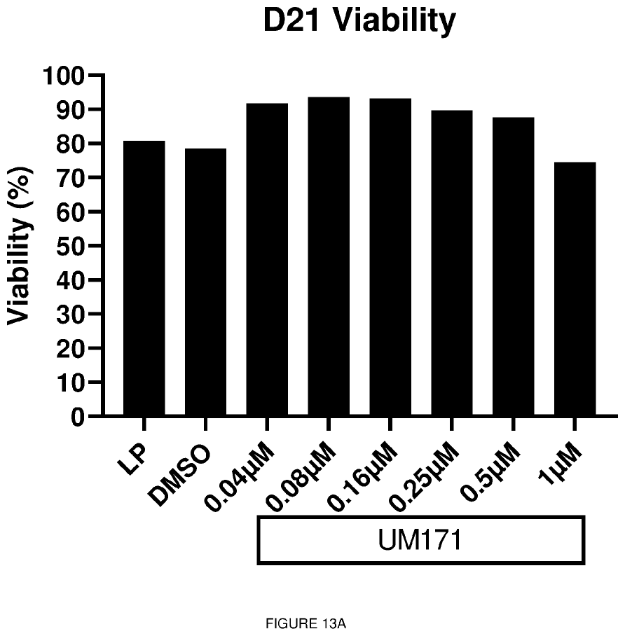

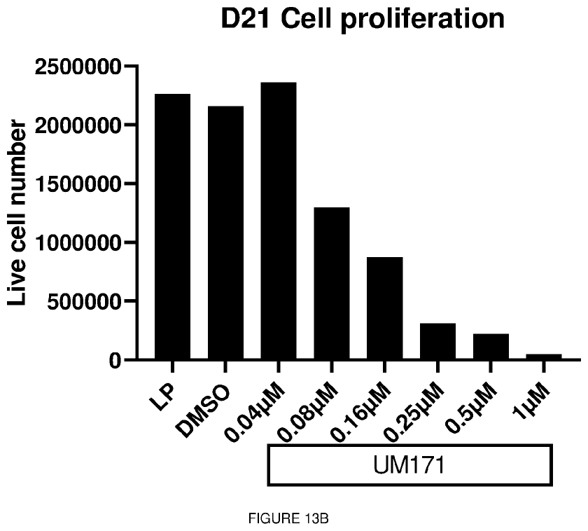

Thawing frozen iPro T cells (stage 4 cells) in stage 5 media led to cell death, however, the addition of UM729 to stage 4 media improved the recovery from freeze/thaw (FIG. 11). After culturing in stage 5 media for 7 days, the cells pre-treated with UM729 at 1 μM presented a higher viability and an increased cell proliferation (FIG. 12).

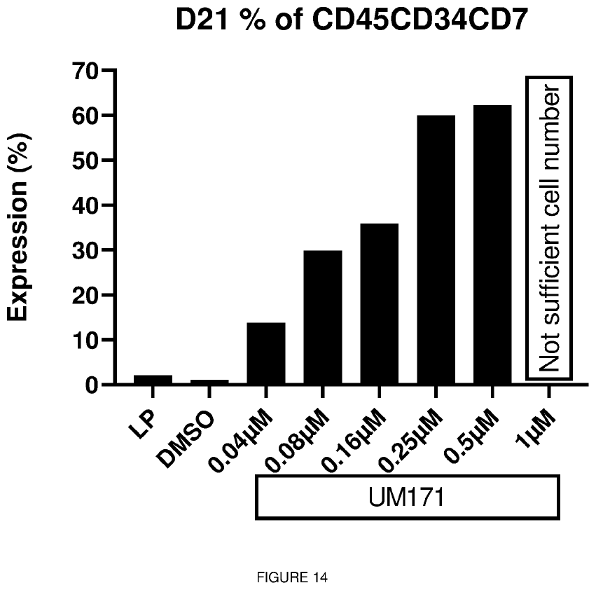

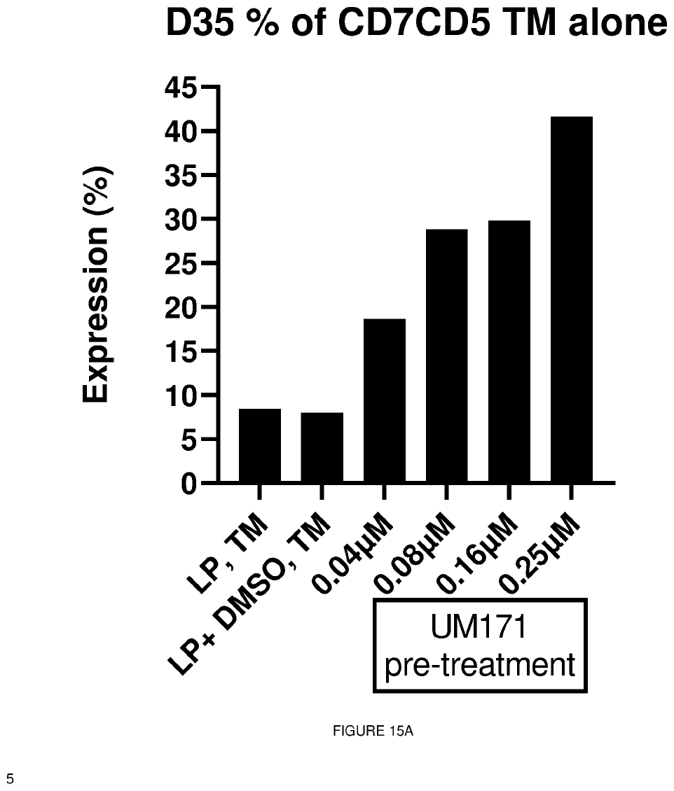

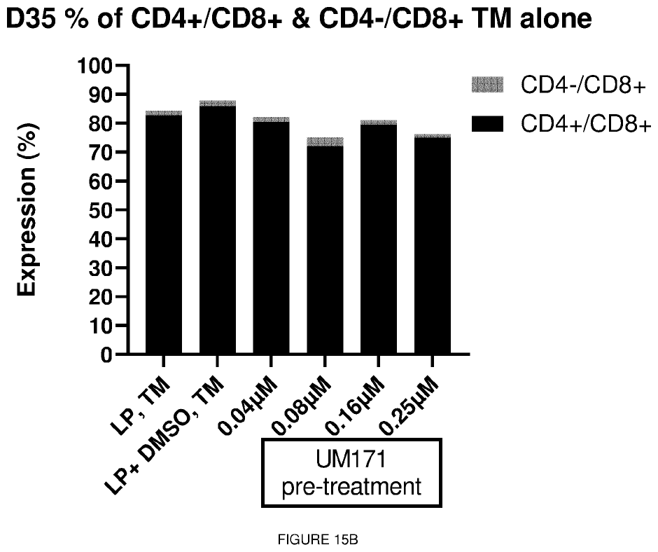

The addition of UM171 did not affect proliferation and viability at low concentrations but was found to cease proliferation of iPSC derived CD34+ cells at the same concentrations used for UM729 (0.25 uM or greater) in stage 4, lymphoid proliferation medium (LP) (FIGS. 13A and 13B). The addition of UM171 to the lymphoid proliferation medium (LP) of stage 4 was also found to enrich CD45+CD34+CD7+ cells (CLPs) in a dose dependent manner (FIG. 14). UM171-treated cells were also shown to progress to CD7CD5 iPro T cells and CD4CD8 DP T cells in stage 5 (FIGS. 15A and 151B).

TABLE 1
List of antibodies and volumes used to identify T cells

| Antibody | Volume/test (μl) | |
|---|---|---|
| TCRαβ (IP26) PE (BioLegend: 306708) | 2.5 | μl |
| TCRγδ (B1) APC (BioLegend: 331212) | 5 | μl |
| CD5 (UCHT2) BV421 (BD: 562646) | 5 | μl |
| CD7 (CD7-6B7) Percp cy5.5 (BioLegend: 343116) | 2.5 | μl |
| CD45 (HI30) BUV395 (BD: 563792) | 2.5 | μl |
| CD4 (OKT4) BV786 (BioLegend: 317442) | 1.25 | μl |
| CD3 (SK7) AF488 (BioLegend: 344810) | 1.25 | μl |
| CD8α (RPA-T8) Pe-cy7 (BD: 557746) | 2.5 | μl |
| CD8β (2ST8.5H7) BV650 (BD:742393) | 5 | μl |
| CD56 (NCAM16.2) BV605 (BD: 562780) | 1 | μl |

| | |
|---|---|
| Ef506 BV510 (Invitrogen: 65-0866-14) | 1/100 dilution |

1. A method of producing a population of progenitor T cells comprising;
differentiating a population of haematopoietic progenitor cells (HPCs) into progenitor T cells in the presence of a pyrimidoindole compound.

2. A method according to claim 1 wherein the presence of the pyrimidoindole compound increases the proportion of HPCs that differentiate into progenitor T cells.

3. A method according to any one of the preceding claims wherein the HPCs are differentiated by a method comprising culturing the population of HPCs in a lymphoid expansion medium supplemented with an effective amount of the pyrimidoindole compound.

4. A method according to claim 3 wherein the lymphoid expansion medium consists of a chemically defined nutrient medium supplemented with effective amounts of SCF, FLT3L, TPO, IL7 and the pyrimidoindole compound.

5. A method according to any one of the preceding claims wherein the HPCs have a CD34+ phenotype.

6. A method according to any one of the preceding claims wherein the HPCs are differentiated by a method comprising differentiating the HPCs into common lymphoid progenitor cells (CLPs) and differentiating the CLPs into progenitor T cells.

7. A method according to any one of the preceding claims wherein the population of HPCs is produced in vitro from induced pluripotent stem cells (iPSCs).

8. A method according to claim 7 wherein the method comprises providing a population of iPSCs and differentiating the iPSCs into a population of HPCs.

9. A method according to any one of the preceding claims wherein the progenitor T cells have a CD5+, CD7+ phenotype.

10. A method for expanding progenitor T cells comprising;
culturing the progenitor T cells in the presence of a pyrimidoindole compound.

11. A method according to claim 10 wherein the presence of the pyrimidoindole compound increases the proliferation of the progenitor T cells in the population.

12. A method according to claim 10 or claim 11 wherein expansion of the progenitor T cells is for at least 21 days

13. A method according to any one of the preceding claims wherein the pyrimidoindole compound is a substituted pyrimido[4,5-b]indole.

14. A method according to claim 13 wherein the pyrimidoindole compound is methyl 4-(3-piperidin-1-ylpropylamino)-9H-pyrimido[4,5-b] indole-7-carboxylate.

15. A method according to claim 13 wherein the pyrimidoindole compound is (1R,4R)—N1-(2-benzyl-7-(2-methyl-2H-tetrazol-5-yl)-9H-pyrimido[4,5-b]indol-4-yl)cyclohexane-1,4-diamine.

16. A method according to any one of the preceding claims, wherein the pyrimidoindole compound is present at a concentration less than 5 μM.

17. A method according to any one of claims 1 to 16 wherein the method further comprises introducing heterologous nucleic acid encoding an αβ TCR into the iPSCs, HPCs or progenitor T cells.

18. A method according to claim 17 wherein the heterologous nucleic acid encoding the αβ TCR is comprised in an expression vector.

19. A method according to claim 18 wherein the expression vector is a lentiviral vector.

20. A method according to any one of claims 17 to 19 wherein the αβ TCR is an affinity enhanced TCR.

21. A method according to one of claims 17 to 20 wherein the αβ TCR binds specifically to an MHC displaying a peptide fragment of a target antigen expressed by cells or specifically binds to a target antigen or peptide thereof expressed by cells independently of MHC presentation.

22. A method according to claim 21 wherein the αβ TCR binds specifically to an MHC displaying a peptide fragment of a tumour antigen expressed by the cancer cells or binds specifically to a tumour antigen or peptide fragment thereof expressed by cancer cells independently of MHC presentation.

23. A method according to any one of the preceding claims comprising further differentiating the progenitor T cells to produce TCR αβ+ T cells.

24. A method according to claim 23 wherein the progenitor cells are further differentiated by a method comprising culturing the population of progenitor T cells in a T cell maturation medium.

25. A method according to claim 23 or claim 24 wherein the TCR αβ+ T cells have a CD8+CD4+ phenotype.

26. A method according to any one of claims 23 to 25 comprising activating and expanding the TCR αβ+ T cells to produce a population of T cells have a CD8+ single positive phenotype or a CD4+ single positive phenotype.

27. A method according to any one of claims 23 to 26 wherein the TCR αβ+ T cells specifically bind to cells expressing a target antigen.

28. A method according to claim 27 wherein the target antigen is a tumour antigen.

29. A method according to claim 28 wherein the TCR αβ+ T cells specifically bind to cancer cells expressing the tumour antigen.

30. A method according to any one of claims 23 to 29 further comprising isolating or purifying the TCR αβ+ T cells.

31. A method according to claim 30 wherein TCR αβ+ T cells are isolated by magnetic activated cell sorting.

32. A method according to according to any one of claims 23 to 31 further comprising concentrating the population of TCR αβ+ T cells.

33. A method according to according to any one of claims 23 to 32 comprising storing the population of TCR αβ+ T cells.

34. A method according to any one of claims 23 to 33 comprising formulating the population of TCR αβ+ T cells with a pharmaceutically acceptable excipient.

35. A population of progenitor T cells produced by a method according to any one of claims 1 to 22.

36. A population of TCR αβ+ T cells produced by a method according to any one of claims 23 to 34.

37. A pharmaceutical composition comprising a population of TCR αβ+ T cells produced by a method according to any one of claims 23 to 34 and a pharmaceutically acceptable excipient.

38. A population of TCR αβ+ T cells produced by a method according to any one of claims 23 to 34 for use in a method of treatment.

39. A population of TCR αβ+ T cells produced by a method according to any one of claims 23 to 34 for use in a method of treatment of cancer.
